(19) United States
(12) Patent Application Publication
(10) Pub. No.: US 2023/0160847 A1
(43) Pub. Date: May 25, 2023

(54) NANOMATERIALS FOR ELECTROCHEMICAL DETECTION OF PHENOLIC ANALYTES

(71) Applicant: EYE3CONCEPTS INC., BURLINGTON (CA)

(72) Inventors: SATISH KUMAR TUTEJA, HAMILTON (CA); SYED RAHIN AHMED, HAMILTON (CA); GRETER AMELIA ORTEGA RODRIGUEZ, HAMILTON (CA); SESHASAI SRINIVASAN, BRAMPTON (CA); AMIN REZA JABZADEH, BURLINGTON (CA)

(21) Appl. No.: 17/919,071

(22) PCT Filed: Apr. 15, 2021
(86) PCT No.: PCT/CA2021/050507

§ 371 Date: Oct. 14, 2022

Related U.S. Application Data
(60) Provisional application No. 63/010,484, filed on Apr. 15, 2020.
(60) Provisional application No. 63/037,252, filed on Jun. 10, 2020.

(51) Int. Cl. G01N 27/327; B82Y 30/00

(52) U.S. Cl. CPC G01N 27/3278; B82Y 30/00; G01N 33/948

(57) ABSTRACT

Composite nanomaterials including a carbon nanomaterial and an electrocatalyst are disclosed and shown to facilitate enhanced detection, via electro-oxidation, of phenolic analytes when applied to a sensing electrode, such as the working electrode of an electrochemical sensor. In other example embodiments, methods and devices for improved electrochemical detection of phenolic analytes are disclosed in which a sensor electrode is modified by the presence of graphene nanosheets. Such modified electrodes may be employed to provide working electrodes in electrochemical sensors for the rapid detection of cannabinoids and/or associated metabolites in saliva. In some example implementations, the nanocomposite or graphene nanosheets are functionalized with magnetic particles and provided in a suspension that is initially contacted with the sample prior to being magnetically drawn to the surface of the electrode for electrochemical processing.

### CROSS-REFERENCE TO RELATED APPLICATION

This application claims priority to U.S. Provisional Patent Application No. 63/010,484, titled “NANOSTRUCTURES EMBEDDED ELECTRONIC SENSING DEVICE FOR ULTRA-LOW LEVEL DETECTION OF THC” and filed on Apr. 15, 2020, the entire contents of which is incorporated herein by reference, and to U.S. Provisional Patent Application No. 63/037,252, titled “NANOSTRUCTURES EMBEDDED ELECTRONIC SENSING DEVICE FOR ULTRA-LOW LEVEL DETECTION OF THC” and filed on Jun. 10, 2020.

### BACKGROUND

Cannabis (common name is marijuana) has been documented as the most widely abused psychoactive drugs worldwide consumed through ingestion or inhalation. Cannabis impaired driving poses a significant threat to road safety. Every year, numerous people are encountered with critical vehicle collisions that lead to accidental death due to marijuana abuse. The number of car accidents has been increased by the influence of marijuana in recent years. Moreover, chronic marijuana consumption leads to addiction and causes many adverse health effects such as respiratory disorders, cardiac disjunction, and mental problems.

To regulate driving under the influence drivers, there is a high requirement for a precise, handy, and instant sensor to evaluate the level of cannabis in drivers at the roadside and other necessary checkpoints. Delta 9-tetrahydrocannabinol (Δ9-THC) is the principal psychoactive molecule present in cannabis that interacts with cannabinoid receptors in the brain. It produces a short term and long-term effects such as alteration in mood, relaxing and overjoyed happiness emotional outcomes, and is used as a biomarker for cannabis detection.

At present, the monitoring of Δ9-THC monitoring in body fluids (saliva, urine, and blood), are conducted by national testing centers and private laboratories using time-consuming protocols and heavy complex instruments based on colorimetry, spectrophotometry, immunoassays, chromatographic and mass spectrometry.

Unfortunately, the above methods require suitable procedures to evaluate in well-equipped laboratory infrastructure and are incompatible with rapid roadside testing. Therefore, a significant demand currently exists for fast (ideally ≥60 seconds), accurate, sensitive (preferably <25 ng/ml), and convenient roadside analysis to evaluate the level of Δ9-THC in influenced drivers. Moreover, such roadside testing devices should be noninvasive, miniature, with instant readout, and portable. Furthermore, roadside testing devices should be able to perform saliva-based Δ9-THC detection, as it is noninvasive mode, has simple sample collection requirements, and ideal for on-site screening compared to blood-based analysis that needs invasive sample collection as cannabis may be present at a quantifiable extent in the saliva and essential media for Δ9-THC monitoring. It is also important that roadside THC tests should be simple to perform by non-laboratory and non-expert people. For this purpose, electrochemical techniques based biosensors are drawing considerable interest in the cannabis finding and recommended choice for the evaluation of Δ9-THC quantification.

Electrochemical based examination testing demonstrates several benefits over traditional central laboratory-based chemical analysis, including high intrinsic sensitivity, accuracy, linearity in a wide range of extents, fast analysis, and low volume of sample prerequisite as compared to immunoassay, chromatographic and spectroscopic techniques. Electrochemical detection through voltammetry and amperometric techniques allow the possibility to apply them for the designing the compact and straightforward portable miniaturize handheld instrument. Voltammetric technology-based assays have recently been successfully employed in the analytical testing of drugs such as ecstasy and cocaine.

Additionally, the introduction of nanomaterials in the electrochemical recognition interfaces results in a major enhancement in terms of specificity, sensitivity, and adaptability because of their exceptional catalytic, electrical, and optical properties. The usage of nanoscale structures in electrochemical devices impacts local electron transfer, resulting in a significant enhancement of peak current and leads to a reduction in the signal to noise ratio. Furthermore, carbon nanotubes (CNTs) present metallic conductivity, high chemical stability, and mechanical strength, a large surface area with improved chemical and physical interaction with analytes.

For these reasons, the enhanced electronic properties of CNTs have been incorporated into electrochemical sensors to decrease overpotential, to increase electroactive surface area, and to allow rapid electrode kinetics. The electrical behavior (resistance/conductance) of such nanostructure-based devices is extremely sensitive to any surface adsorption/perturbation. It is proportional to the direct analyte concentration, which can act as a sensing mechanism for electrochemical-based sensor electrodes.

Despite these early implementations involving the incorporation of CNTs into electrochemical sensors, an electrochemical-based sensor for the detection of THC with sufficient sensitivity remains elusive. Accordingly, there remains a strong need for a roadside sensor capable of performing THC detection with a limit of detection less than about 5 ng/mL (16 nM) of Δ9-THC in saliva.

### SUMMARY

Composite nanomaterials including a carbon nanomaterial and an electrocatalyst are disclosed and shown to facilitate enhanced detection, via electro-oxidation, of phenolic analytes when applied to a sensing electrode, such as the working electrode of an electrochemical sensor. In other example embodiments, methods and devices for improved electrochemical detection of phenolic analytes are disclosed in which a sensor electrode is modified by the presence of graphene nanosheets. Such modified electrodes may be employed to provide working electrodes in electrochemical sensors for the rapid detection of cannabinoids and/or associated metabolites in saliva. In some example implementations, the nanocomposite or graphene nanosheets are functionalized with magnetic particles and provided in a suspension that is initially contacted with the sample prior to being magnetically drawn to the surface of the electrode for electrochemical processing.

Accordingly, in a first aspect, there is provided a method of performing an electrochemical assay to detect an analyte comprising an oxidizable phenolic group, the method comprising:

contacting a sample suspected of containing the analyte with a modified electrode, wherein the modified electrode comprises a nanocomposite, the nanocomposite comprising a carbon nanomaterial and an electrocatalyst;

incubating the sample with the modified electrode;

applying a potential suitable for electrochemically oxidizing the oxidizable phenolic group of the analyte; and

detecting an assay signal associated with electrochemical oxidation of the analyte.

In some example implementations of the method, the carbon nanomaterial comprises carbon nanotubes.

In some example implementations of the method, the carbon nanomaterial comprises graphene.

In some example implementations of the method, the carbon nanomaterial comprises one or more of graphene quantum dots, fullerenes, and carbon nanoribbons.

In some example implementations of the method, the electrocatalyst comprises ferrocene, ferricyanide, and/or derivatives thereof.

In some example implementations of the method, the electrocatalyst comprises any one or more of metal oxide frameworks, metal and metal oxide nanoparticles, Prussian Blue nanoparticles, polymer-metal complexes (ruthenium, iron, manganese) nanoparticles, and dendrimers.

In some example implementations of the method, the assay signal is processed to infer a concentration of the analyte in the sample.

In some example implementations of the method, the analyte is a cannabinoid or a metabolite thereof.

In some example implementations of the method, the cannabinoid is delta 9-tetrahydrocannabinol.

In some example implementations of the method, the analyte is one of an opiate, a neurotransmitter, a hormone, or a metabolite thereof.

In some example implementations of the method, the sample is saliva.

In some example implementations of the method, the assay signal is obtained after an incubation delay of less than 3 minutes, 2 minutes, or less than or equal to 1 minute.

In some example implementations of the method, one or more assay parameters of the electrochemical assay are configured such that a limit of detection of the electrochemical assay lies between approximately 2 ng/ml and 10 ng/ml.

In some example implementations, the method further comprises applying a pre-conditioning potential to the modified electrode prior to detecting the assay signal.

In some example implementations of the method, the assay signal is obtained by performing a voltammetric measurement.

In some example implementations, the method further comprises, prior to contacting the sample with the modified electrode: contacting the sample with a suspension comprising capped magnetic particles, the capped magnetic particles comprising a charged polymeric shell, thereby forming a mixture; incubating the mixture for a time duration sufficient to facilitate adsorption of polar interferents within the sample onto the capped magnetic particles; and employing a magnetic field to separate the capped magnetic particles from the mixture, thereby reducing a concentration of the polar interferents within the sample.

In another aspect, there is provided a method of performing an electrochemical assay to detect a cannabinoid analyte, the cannabinoid analyte comprising delta 9-tetrahydrocannabinol or a metabolite thereof, the method comprising:

contacting a saliva sample suspected of containing the cannabinoid analyte with a modified electrode, wherein the modified electrode comprises graphene nanosheets;

incubating the saliva sample with the modified electrode for a time duration of less than 5 minutes;

applying a potential suitable for electrochemically oxidizing the cannabinoid analyte; and

detecting an assay signal associated with electrochemical oxidation of the cannabinoid analyte.

The method may further include applying a pre-conditioning potential to the modified electrode prior to detecting the assay signal.

In another aspect, there is provided a method of performing an electrochemical assay to detect an analyte comprising an oxidizable phenolic group, the method comprising:

contacting a sample suspected of containing the analyte with a suspension comprising a magnetic nanocomposite, the magnetic nanocomposite comprising a carbon nanomaterial and magnetic particles, thereby obtaining a mixture;

incubating the mixture;

applying a magnetic field configured to contact the magnetic nanocomposite with a surface of an electrode;

applying a potential to the electrode, the potential being suitable for electrochemically oxidizing the oxidizable phenolic group of the analyte; and

detecting an assay signal associated with electrochemical oxidation of the analyte.

The magnetic nanocomposite may further include an electrocatalyst. The electrocatalyst may include ferrocene, ferricyanide, and/or derivatives thereof.

In some example implementations of the method, the carbon nanomaterial comprises carbon nanotubes. In some example implementations of the method, the carbon nanomaterial comprises graphene nanosheets.

In another aspect, there is provided a method of modifying an electrode to incorporate a nanocomposite, the method comprising:

providing suspension comprising the nanocomposite, the nanocomposite comprising nanocomposite comprising a carbon nanomaterial and an electrocatalyst;

drop casting the suspension onto the electrode; and

incorporating the nanocomposite onto the electrode via electrodeposition.

In another aspect, there is provided an electrochemical sensor for detecting a presence of a cannabinoid analyte in a sample, the cannabinoid analyte comprising delta 9-tetrahydrocannabinol or a metabolite thereof, the electrochemical sensor comprising a working electrode modified with a nanocomposite, the nanocomposite comprising a carbon nanomaterial and an electrocatalyst configured to catalyze electrochemical oxidation of a phenol group of the cannabinoid analyte.

The electrochemical sensor may further include control and processing circuitry operatively coupled to the working electrode, the control and processing circuitry comprising at least one processor and associated memory, the memory being programmed with instructions executable by the at least one processor for performing operations comprising:

performing a voltametric scan to obtain an assay signal associated with oxidation of a phenolic analyte at the working electrode, the oxidation being catalyzed by the nanocomposite; and

processing the assay signal to infer a concentration of the phenolic analyte in according to calibration data stored in the memory.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1A illustrates a schematic representation of the composite deposited on Fe@CNT/SPE and the electrocatalytic effect of ferricyanide.

FIGS. 1B and 1C schematically illustrate the THC electro-oxidation process for the example case of a nanocomposite formed by carbon nanotubes (CNTs) and ferrocene.

FIG. 1D is a diagram showing the magnetic nanoparticle approaches to eliminate interferences from the saliva sample before THC detection (from 1 to 5) and to concentrate the THC molecules on the WE area before the electrochemical measure (from 6 to 10).

FIGS. 2A and 2B illustrate electrochemical label-free THC sensing using graphene immobilized nanostructures.

FIGS. 3A and 3B illustrate example sensor configurations for performing electrochemical detection of phenolic analytes using a modified electrode.

FIG. 3C is an example system for performing electrochemical detection of phenolic analytes using a modified electrode.

FIGS. 4A-4D show (FIG. 4A) the CV curves at each step of electrode modification before and after CNT nanocomposite modification of bare SPE, recorded at a fixed scan rate of 50 mV s−1 in a redox probe solution of 5 mM of ferrocyanide and 5 mM of ferricyanide prepared in 200 mM PBS buffer pH 7.4; (FIG. 4B) CNT stability of SPE (FIG. 4C) Electrodeposition of CNT ferrocene composite and stability of immobilized Nanocomposite nanostructures studies using several scans of continuous CV cycles; (FIG. 4D) Effect of scan rate on cyclic voltammogram of CNT ferrocene nanocomposite immobilized electrode.

FIGS. 5A-5C show SEM micrographs at different stages, where FIG. 5A shows bare screen printed electrodes (SPEs), FIG. 5B shows carbon-nanotube (CNT) modified screen-printed electrodes, and FIG. 5C shows CNT ferrocene nanocomposite modified screen-printed electrodes.

FIGS. 6A-6D show EDX analysis for (FIG. 6A-6B) CNT modified screen-printed electrodes and (FIG. 6C-6D) CNT ferrocene nanocomposite modified screen-printed electrodes.

FIGS. 7A and 7B show (FIG. 7A) UV spectral analysis of pristine, ferrocene, and CNT-ferrocene modified screen-printed electrodes and (FIG. 7B) Raman spectral characterization of bare SPE, pristine CNT, and CNT ferrocene nanocomposite modified screen-printed electrodes.

FIGS. 7C and 7D plot (FIG. 7C) results from electrode stability testing of CNT-ferrocene modified screen-printed electrodes using CV and (FIG. 7D) the cathodic current over 10 CV cycles to check the stability and irreversibility of nanocomposite modified electrodes.

FIG. 7E shows results from a pH study of THC sensing using CNT-Ferrocene nanocomposite modified SPE.

FIGS. 8A-8D demonstrate the electrochemical sensing of THC using (FIG. 8A) SWV and (FIG. 8B) chrono amperometric detection in optimized PBS buffer at pH 7.4 using CNT ferrocene nanocomposite modified screen-printed electrodes, (FIG. 8C) the standard SWV calibration plot for the CNT ferrocene nanocomposite SPE against varying concentrations of THC in standard buffer PBS (pH 7.4); (FIG. 8D) amperometric detection calibration plot against different concentrations of THC in simulated Saliva (pH 7.4); each experimental data point represents the average of three independent measurements at separate electrodes, and error bars indicate the standard deviation of the mean (n=3).

FIG. 9 shows investigations of sensor response towards THC, in addition to other non-specific analytes to assess device specificity (concentration was fixed 100 ng/mL).

FIG. 10 shows the storage stability and shelf life of CNT-ferrocene nanocomposite modified SPE based developed nanointerface.

FIG. 11 is a table presenting the analysis of standard Buffer, artificial spiked Saliva (containing known THC concentrations) using SWV technique. (Linear Equation y=0.0047x+0.1484 R²=0.8861, calculated from the calibration plot of THC in standard PBS buffer, pH 7.4).

FIG. 12 shows SEM images employed for characterization of exfoliated electrodeposited graphene nanostructures over the SPE; inset shows the a magnifyied view of graphene nanostructures.

FIGS. 13A and 13B plot (FIG. 13A) the CV curves before and after graphene modification of Bare SPE, recorded at a fixed scan rate of 50 mV/s in a redox probe solution of 5 mM of ferrocyanide and 5 mM of ferricyanide prepared in 200 mM PBS buffer pH 7.4; (FIG. 13B) electrodeposition of graphene and stability of immobilized graphene nanostructures studies using 10 continuous CV cycles.

FIG. 13C plots the cathodic current over 10 CV cycles to verify the stability and irreversibility of electrodeposition of graphene.

FIG. 14 plots the results from studies of buffer optimization.

FIGS. 15A and 15B plot (FIG. 15A) square wave voltammogram of THC at different pH of PBS using graphene immobilized SPE; (FIG. 15B) shows the effect of scan rate on cyclic voltammogram of graphene immobilized electrode.

FIG. 16 plots the results from studies of THC incubation time.

FIGS. 17A and 17B plot results from electrochemical sensing of THC using (FIG. 17A) SWV and (17B) chrono amperometric detection in optimized PBS buffer at pH 7.4 using graphene nanostructures modified screen-printed electrodes.

FIGS. 18A-18D plot (FIG. 18A) the SWV response of eGr/SPE measured against varying concentrations THC diluted in simulated saliva (pH 7.4); (FIG. 18B) the standard SWV calibration plot for the of eGr/SPE against varying concentrations of THC in simulated saliva (pH 7.4); (FIG. 18C) the standard SWV calibration plot for the of eGr/SPE against varying concentrations of THC in PBS Buffer (pH 7.4); (FIG. 18D) chronoamperometric detection (AD) curve of eGr/SPE tested with varying concentrations of THC diluted in PBS (pH 7.4); (FIG. 18E) amperometric detection calibration plot against varying concentrations of THC in simulated saliva (pH 7.4); (FIG. 18F) amperometric detection calibration plot against varying concentrations of THC in PBS Buffer (pH 7.4). Note: each experimental data point represents the average of three independent measurements at different electrodes, and error bars indicate the standard deviation of the mean (n=3).

FIG. 19 is a table presenting an analysis of standard buffer, artificial spiked saliva (containing known THC concentrations) using SWV technique. (Linear Equation y=0.0084x+0.0863, R²=0.9925 calculated from the calibration plot of THC in standard PBS Buffer, pH 7.4).

FIG. 20 plots results of investigations of sensor response towards THC, in addition to other non-specific analytes to assess device specificity (concentration was fixed 100 ng/mL).

FIG. 21 plots results from studies of storage stability and shelf life of eGr/SPE based developed nanointerface.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

As used herein, the phrases “carbon nanostructure” and “carbon nanomaterial” generally refer to a material having at least one dimension in the nanoscale and being formed primarily from carbon. carbon nanostructures comprised of a single sheet of pure graphitic layers (hexagonal lattice of carbon sheets called graphene layers), having diameters as small as 1 nm. Carbon nanomaterials include forms of graphitic carbon having conjugated, repeating, aromatic carbon rings, including, but not limited to, carbon nanotubes (CNTs), including single and multi-wall CNTs, graphene, graphene quantum dots QDs, fullerenes, and nanoribbons.

As used herein, the phrase “electrocatalyst” refers to a catalyst which aids in the electrooxidation in an electrochemical reaction.

As used herein, the phrase “drop cast” refers to a method of depositing nanoparticles into a thin film. The nanoparticles are first mixed in a solvent and then dropped onto a substrate. As the solvent evaporates, the nanoparticles are deposited onto the surface of the substrate.

The present inventors, aware of the limitations of existing carbon-nanomaterial-based approaches to the electrochemical detection of THC, sought an improved detection platform that would facilitate an improved limit of detection without sacrificing the ability to provide a rapid time to result and achieve long-term stability. As described below, through experimentation, the present inventors identified several new avenues to enhanced THC detection using modified carbon nanomaterials.

### Carbon Nanomaterial and Electrocatalyst Nanocomposites for Improved Electrochemical Detection of Phenolic Analytes

The present inventors realized that by combining an electrocatalyst with a carbon nanomaterial to form a nanocomposite, and applying the nanocomposite to an electrode, the synergistic effect of the catalytic activity of the electrocatalyst and the enhanced conductivity of the carbon nanomaterial can enhance the electrochemical oxidation of phenols and lead to improved detection of phenolic (phenol-containing) analytes such as THC. Accordingly, in some aspects of the present disclosure, composite nanomaterials including a carbon nanomaterial and an electrocatalyst are disclosed and are shown to facilitate enhanced detection of phenolic analytes when applied to a sensing electrode, such as the working electrode of an electrochemical sensor. Such nanocomposites may be obtained by combining carbon nanomaterials that increase the electroactive area of an electrode (e.g. a working electrode of an electrochemical sensor) and also promote the interaction of THC (or other phenol-containing analytes) with the electrocatalyst species that function as mediators between the THC molecules and the electrode during the electro-oxidation.

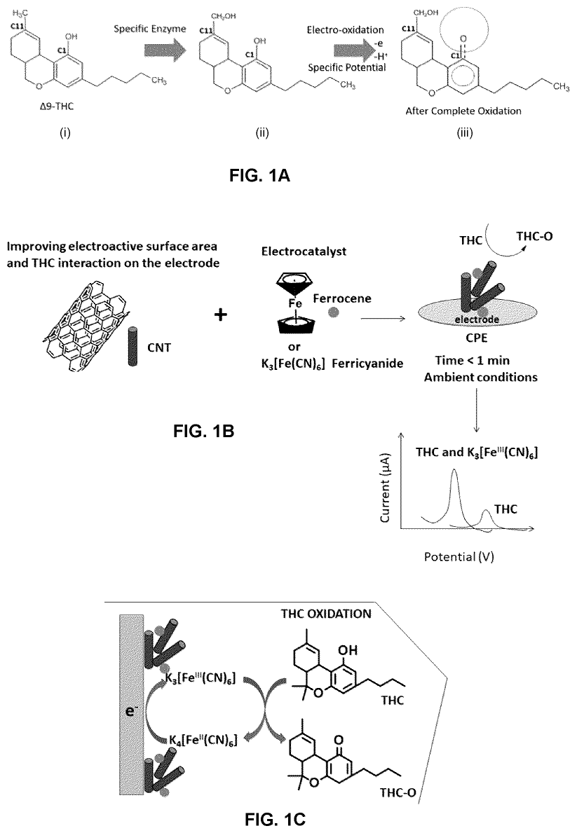

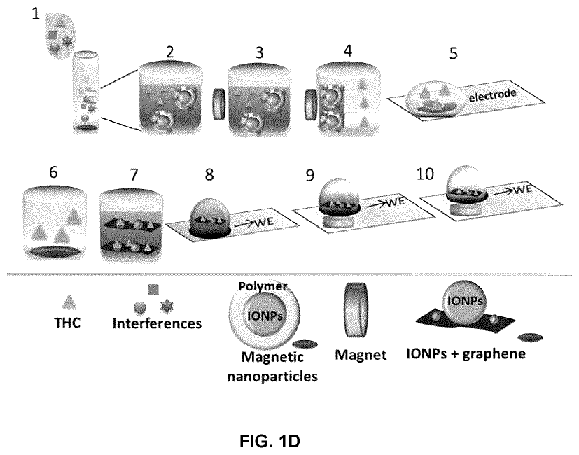

FIG. 1A illustrates the principle of the enhancement of electrical detection sensitivity to phenolic analytes via the synergistic effect of a nanocarbon/electrocatalyst nanocomposite. The nanocomposite, which resides on a sensor electrode and includes a carbon nanostructure and an electrocatalytic enzyme (not shown in the figure), efficiently catalyzes the C-11 hydroxylation of THC and thus boosts the electro-oxidation of hydroxyl groups attached to C-1 and C-11 of -THC chemical structure, as shown in steps (i) and (ii) of the illustrated catalytic reaction.

Without intending to be limited by theory, it is believed that when a suitable electro-oxidation potential is applied to the modified sensor electrode, the THC molecule is firstly oxidized to its initial deprotonated intermediary stage and then forms a second intermediate having a phenoxide anion. The complete electro-oxidation, shown in step (iii), generates phenoxy radicals. These phenoxy radicals can interact with other THC molecules to initiate the oxidation of another phenolic structure present in the sample, thereby further enhancing the complete oxidation and electrochemical detection of Δ9-THC. It is therefore believed that the THC molecules undergo complete oxidation on the surface of the modified electrode by synergistic functionality of the enzyme and the application of the oxidation potential. Accordingly, the enzyme aids in specificity as well as complete oxidation of THC, which can lead to ultra-sensitivity of the THC assay.

This reaction is illustrated in FIG. 1B in the example case in which the carbon nanomaterial is a carbon nanotube (CNT) and the electrocatalyst is ferrocene or ferricyanide. As shown in the figure, a nanocarbon-electrocatalyst nanocomposite is formed by depositing ferrocene or ferricyanide onto CNTs such that the nanocomposite resides on the sensing electrode (e.g. a working electrode of an electrochemical sensor). The nanocomposite catalyzes the oxidation of THC when a suitable potential is applied to the electrode, and the assay signal, for example, in the form of a CV graph, is obtained after a short incubation, such as less than one minute as shown in the figure.

FIG. 10 illustrates the role of the nanocomposite in facilitating the electro-oxidation of THC in the example case of ferricyanide as the electrocatalyst. The application of a potential of 0.4V to 0.6V to a sample contacting the modified electrode causes the THC molecule in the sample to oxidize. This oxidized form of THC on the modified electrode generates a unique signal at specific potential, as shown in the CV graph. The peak potential arises due to the electrochemical response during the electro-oxidation of the phenol functional group in the molecular structure of the THC. The intensity of the oxidation current peak is directly proportional to the concentration of THC and can be measured by electrochemical techniques such as square wave voltammetry (SWV), or other voltametric modalities described in further detail below. The current intensity (measured, for example, by a potentiostat) is correlated to the THC concentration present in the sample (e.g. via a predetermined calibration curve or dataset). As can be seen in the figure, the close proximity of the electrocatalyst to the electrode surface and the enhancement of the electrode surface conductivity and surface area via the carbon nanotubes leads to an enhanced electro-oxidation of the phenol group of the THC molecule.

Without intending to be limited by theory, it is believed that the improvement in the redox reaction proximal to the nanocomposite/electrode is also due to both the electrocatalytically properties of ferricyanide and also the inherent redox characteristics of the CNT, which facilitates electrochemical redox reactions due to its high porosity and its specific interface surface. While FIGS. 1A-1C illustrate the enhanced electro-oxidation of THC, it will be understood that THC is merely employed as an example of a phenolic analyte and that other phenolic analytes may be detected in the alternative

It will be understood that the nanocomposite may be formed according to a wide variety of carbon nanomaterials and electrocatalysts. Non-limiting examples of carbon nanomaterials include, but are not limited to carbon nanotubes (CNTs), such as multi-wall CNTs, graphene, graphene quantum dots QDs, fullerenes, and nanoribbons. Non-limiting examples of electrocatalysts include ferrocene and derivates, ferricyanide, Prussian Blue nanoparticles, metal complexes (ruthenium, iron, manganese) nanoparticles, dendrimers, platinum nanoparticles, palladium nanoparticles, and gold nanoparticles.

As described in detail below, various voltametric experiments were performed to study the electrochemical behavior of the electrocatalyst trapped by physical interactions such as strong hydrophobic forces between the electrocatalyst and the carbon-based nanostructures structure immobilized on an electrode, as well as its interaction with one of its substrates, THC. The present inventors found that electrochemical reactions performed according to the present example embodiments can facilitate the accurate and rapid detection of THC and provide an electrochemical sensing platform that is stable when stored in dry conditions.

### Graphene Nanosheets for Improved Electrochemical Detection of Phenolic Analytes

The present inventors, through further experimental investigation, determined that sensor electrodes modified with graphene nanosheets also provided improved electrochemical detection of THC, and more generally, phenolic analytes. Indeed, the present inventors found that exfoliated 2D graphene nanosheets, when immobilized (or magnetically captured, as described further below) on a sensor electrode (such as a screen-printed electrode), resulted in improved amplification of electrochemical signals associated with the oxidation of phenolic analytes, the enhancement of surface loading, and minimization and/or reduction of background noise signals. These improvements are demonstrated in various examples provided below.

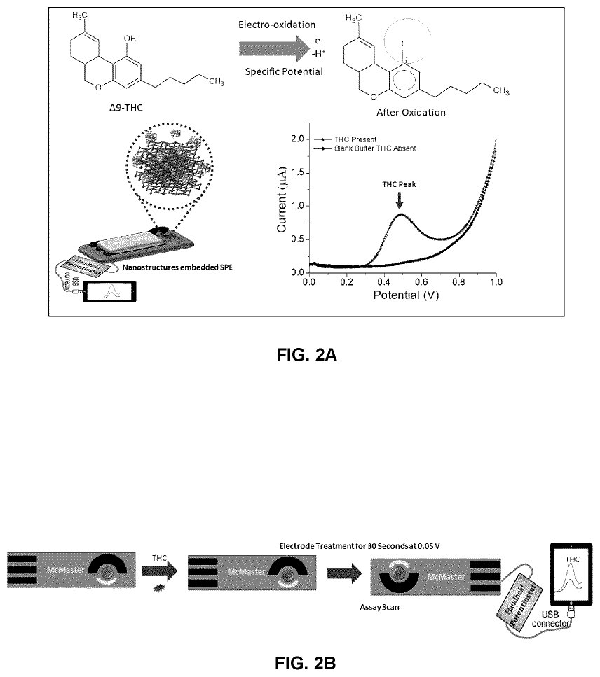

FIG. 2A schematically illustrates the electro-oxidation of THC by an electrode modified with graphene nanosheets, in which a single electro-oxidation peak is detected in the CV scan. This example embodiment facilitates a label-free approach to the electrochemical detection of THC without requiring additional reagents such as antibodies, enzymes, or other labeling agents.

Experiments related to the generation of graphene nanosheets by exfoliation, electroreduction and deposition of graphene nanosheets on screen printed electrodes, and experimental demonstrations of the electrochemical performance of graphene-nanosheet-modified electrodes, are described in further detail below. Electrochemical techniques are employed to characterize graphene-nanosheet-modified electrodes, and the results presented in the examples below indicate the successful exfoliation and irreversible electrodeposition of graphene nanosheets over the working electrode. Indeed, the experiments summarized in the examples below were performed in the absence of cross-linker moieties, labels, and harmful chemicals.

The resulting graphene-nanosheet-modified screen-printed-electrodes demonstrated high sensitivity for the detection of THC in saliva at low concentrations, high stability, portability, and may be provided in the form of disposable cartridges for electrochemical detection of THC via a portable reader, optionally for use in roadside testing.

### Methods of Preparation of Carbon Nanomaterial and Electrocatalyst Nanocomposites

In some example implementations, a carbon nanomaterial/electrocatalyst nanocomposite may be prepared in the form of a concentrated suspension as follows. Carbon nanomaterials (e.g. MWCNTs) may be dispersed with an electrocatalyst or electrocatalyst precursor (e.g. ferrocene carboxylic acid) in a solvent (e.g. dimethylformamide and DI water (90% DMF:10% DI water)) to form a dispersion. The mixture may be dispersed, for example, using an ultrasonic bath and optionally heated during the ultrasonic dispersing step. The concentration of carbon nanomaterials in the mixture may range, for example, between 1 mg/mL to 5 mg/mL and the concentration of electrocatalyst in the mixture may range, for example, between 0.5 mg/mL to 2.5 mg/mL. The resulting dispersion may subsequently be centrifuged, for example, within 4000 to 12000 RPM for 10 to 30 minutes. The resultant solid after the centrifugation is resuspended in the 90% DMF:10% DI water with a 1 mg/mL concentration.

### Methods of Preparation of Graphene-Nanosheets

In some example implementations, graphene nanosheets may be prepared as follows. Graphene nanosheets may be exfoliated by ultrasonication, for example, in a 50:50 (DMF:H2O) dispersion. The concentration of graphene nanosheets in the dispersion may range from 1 mg to 10 mg/mL. The dispersion may subsequently be centrifuged, for example, within 4000 rpm to 12000 rpm for 10 to 30 minutes. In the present example, N,N-Dimethylformamide (DMF)/water in the ratio of 50%:50% (v/v) as an organic mixture was found to be beneficial in generating a stable graphene dispersion because of its electrochemical steadiness behavior and also compatibility for the THC molecule in case any residues remain after drying.

### Methods of Fabrication of Electrodes Modified with Carbon Nanomaterial and Electrocatalyst Nanocomposites and Graphene Nanosheets

In some example implementations involving electrical sensing, an electrode, such as a working electrode in the case of an electrochemical testing device, may be modified by the incorporation of a carbon nanomaterial/electrocatalyst nanocomposite and/or graphene nanosheets.

The electrode that is to be modified may be made from any suitable conductive material. In one embodiment, the electrode may include a carbon-based material, a nanomaterial, a metal-based material, or a combination thereof. In one embodiment, the electrode may include carbon, gold, platinum, palladium, ruthenium, rhodium, or a combination thereof. In a further example implementation, the electrode may include a screen-printed electrode (SPE). The working electrode may be provided in any suitable shape or size. Examples of SPEs include, but are not limited to, a Zensor electrode, a Dropsens electrode, and a Kanichi electrode.

The modified sensor can be fabricated using any suitable process capable of associating the carbon nanomaterial and electrocatalyst nanocomposite and/or graphene nanosheets with the electrode surface. The electrode may be polished and/or washed prior to the deposition of the carbon nanomaterial and electrocatalyst nanocomposite and/or graphene nanosheets. The electrode may be washed after the deposition of the carbon nanomaterial and electrocatalyst nanocomposite and/or graphene nanosheets.

In some example implementations, an electrode may be modified by drop-casting a suspension of synthesized carbon nanomaterial/electrocatalyst nanocomposite and/or graphene nanosheets onto the electrode surface (e.g. approximately 1 uL of 1 mg/mL concentration), annealing the modified electrode, and performing electrodeposition. Example annealing conditions include 120 to 200° C. for 1 to 3 hours. The suspension that is contacted with the electrode during the deposition process may include an aqueous solvent, an organic solvent, or a mixture thereof.

In some example implementations, the carbon nanomaterial/electrocatalyst nanocomposite and/or graphene nanosheets can be adhered to electrode via electrodeposition.

In embodiments in which electrodeposition is employed to associate the carbon nanomaterial and electrocatalyst nanocomposite with the electrode surface, the electrode can receive an electrolytic solution (which can be, without limitation, a buffer, such as, for example a phosphate buffered saline).

In some embodiments, the carbon nanomaterial/electrocatalyst nanocomposite and/or graphene nanosheets can be adhered to the electrode through electrodeposition by the application of applying at least one potential to the electrode. In a specific example implementation, a plurality of potentials (e.g., a potential scan) can be applied to the electrode in contact with the carbon nanomaterial/electrocatalyst nanocomposite and/or graphene nanosheets. In still another example implementation, a voltammetry technique can be applied to the electrode in contact with the carbon nanomaterial/electrocatalyst nanocomposite and/or graphene nanosheets to facilitate deposition.

In one example implementation, electrodeposition may be performed by applying a plurality of cycles of cyclic voltammetry (CV) (e.g. in the −1 to +1 V potential range, at a scan rate of 0.1 to 0.25 V/s.

In some example implementations involving the deposition of graphene nanosheets, reduction electrochemical scans may be applied, prior to electrodeposition, using linear sweep voltammetry (LSV) techniques at a scan rate of 0.1 to 0.25 V/s V/s (e.g. with a step potential of 0.001 V) in the range of 0 to 1.4 V.

In some example implementations, the entire surface of the electrode (e.g. a working electrode) may be contacted with the carbon nanomaterial/electrocatalyst nanocomposite and/or graphene nanosheet suspension, while in other example implementations, only a portion of the surface of the electrode may be contacted with the carbon nanomaterial/electrocatalyst nanocomposite and/or graphene nanosheet suspension. In some example implementations, two or more layers of the carbon nanomaterial/electrocatalyst nanocomposite and/or graphene nanosheets may be deposited onto the electrode.

### Example Electrochemical Detection Devices

In various example embodiments, improved electrochemical detection devices are provided by employing a sensing electrode (e.g. a working electrode of an electrochemical sensing device) that is modified according to the example methods described above (modified with a carbon nanomaterial/electrocatalyst nanocomposite and/or graphene nanosheets).

An example embodiment of a sensing device 100 including a modified electrode 102 is shown in FIG. 2A. The modified electrode 102 may be a working electrode of an electrochemical sensor. In the embodiment presented in this figure, the modified electrode 102 has been modified with one or more layers of carbon nanomaterial/electrocatalyst nanocomposite and/or graphene nanosheets. The modified electrode 102 can be provided on a substrate 108. The substrate 108 can be an insulated substrate. It is possible that the modified electrode 102 can be self-supporting and as such the substrate 108 may be omitted. A connection 106 connects the modified electrode 102 to the edge of the substrate 108 or to a contact surface or connecting pad (not shown in the figure). In the sensor 100 shown in FIG. 2A, a sample receiving region 110 is in fluid communication with the modified electrode 102. For example, the sample region 110 may be defined to allow contact between the sample and the sensing electrode 102. It will be understood that the sample receiving region 110 does not need to cover the modified electrode 102, in part or in whole (as shown in FIG. 2A), as other configurations for providing fluid communication between the sample receiving region 110 and the sensing electrode 102 can be used (a microfluidic channel for example).

In some example implementations, an electrochemical sensing device may include multiple modified working electrodes. The multiple working electrodes may have the same modified working electrode structure (e.g. for performing multiple tests in parallel) or may have one or more different modified electrodes, where at least two of the modified electrodes may be configured to catalyze different electrochemical reactions.

In some example implementations, the sensor includes one or more reference electrodes. A reference electrode may be associated with one or more working electrodes of a sensing device. The reference electrode is an electrode with a stable and well-defined electrochemical potential against which the potential of the working electrode(s) can be controlled and measured. When the reference electrode is in use, it is intended to be covered by the sample. In one embodiment, the reference electrode comprises or consists of silver. In some example implementations involving a screen-printed reference electrode, the reference electrodes maybe prepared with Ag/AgCl ink or Ag ink.

In some example embodiments, the sensor includes one or more counter electrodes. In an embodiment, each working electrode can be associated with one counter electrode. In another embodiment, two or more working electrodes can be associated with the same counter electrode. The counter electrode completes the circuit of a three-electrode cell, as it allows the passage of current. After the sample is placed on a sample receiving region, a potential is applied between the working electrode and the reference electrode, and the current induced is measured. At the same time, a potential between the counter electrode and the reference electrode is induced which will generate the same amount of current (reverse current). Therefore the working electrode, reference electrode, and counter electrode are all intended to be in fluid communication with the sample. The counter electrode can be made of the same materials as the working electrode and/or the reference electrode. In one example, the counter electrode comprises or consists of carbon ink or platinum.

FIG. 2B illustrates an example implementation of a sensor device that includes 100 that includes a modified working electrode 102, a reference electrode 116, a counter electrode 118. The modified working electrode 102, the reference electrode 116 and the counter electrode 118 are provided on the same substrate 108. The substrate 108 can be insulated. It is understood that any of the electrodes of the sensor 100 can be self-supporting and do not need to be provided on the substrate 108. A connection 106a connects the sensing electrode 102 to a contact surface 120a. A connection 106b connects the reference electrode 116 to a contact surface 120b. A connection 106c connects the counter electrode 118 to a contact surface 120c. A common sample receiving region 110 is provided for all of the electrodes 102, 116, 118. It will be appreciated that the sample receiving region does not need to cover the regions defined by the electrodes, in part or in whole, as other configurations for providing the sample to the electrodes 102, 116, 118 can be designed (a microfluidic channel for example). Distinct sample receiving regions can also be provided for each electrode 102, 116, 118.

### Example Voltametric Detection Methods Employing Modified Working Electrodes having Carbon Nanomaterial/electrocatalyst Nanocomposite and/or Graphene Nanosheets

In some embodiments, an electrochemical sensor having a modified working electrode according to the example methods disclosed above (modified with a carbon nanomaterial/electrocatalyst nanocomposite and/or graphene nanosheets), or variations thereof, may be employed for the detection of an analyte using a voltammetry technique. Voltammetry techniques are electroanalytical techniques based on the detection and quantification of an analyte, by measuring a current as an applied potential is varied. Non-limiting examples of voltametric methods include cyclic voltammetry (CV), linear sweep voltammetry (LSV), differential pulse voltammetry (DPV), and square wave voltammetry (SWV). CV is performed by cycling the potential of a working electrode ramped linearly versus time and measuring the resulting current. LSV measures the current at the working electrodes while the potential between the working electrode and a reference electrode is swept linearly in time. In the DPV technique a potential scan is recovered by imposing potential pulses with a constant amplitude. SVVV is a large-amplitude differential technique in which a waveform composed of a symmetrical square wave, superimposed on a base staircase potential, is applied to the working electrode.

### Magnetic-Particle-Based Removal of Interferents from Saliva

Saliva is a complex bodily fluid containing water, inorganic ions, small organic molecules (some of them are electroactive), and a variety of proteins. The viscosity of human saliva is around 1.30 times higher than water, affecting the diffusion of the analytes as well as the reaction rates on the electrodes. Also, a variety of components of saliva may interfere with the electrochemical performance of the analyte of interest. Considering that THC detection is based on the oxidation signal at around 0.4 V, some of the natural components of saliva (e.g. uric acid, bilirubin, glutamate, cortisol, ascorbic acid, and enzymes) may lead to unwanted effects during measurements due to collateral oxidation reactions at potentials near 0.4 V. These natural components are herein referred to as “interference molecules”. To avoid the side effects of such interferences, and to increase the specificity of the test, the following example separation method was developed.

The electrochemical response of the THC and the interference molecules can be modulated by the optimization of the working electrode composition and the operating parameters (e.g. electrochemical technique, pH, scan rate, deposition time, deposition potential). The chemical structure of the majority of the natural components in saliva presents higher molecular polarity than THC molecules, resulting in different diffusion capacity and reaction rate compared to those of THC. Therefore, optimizing the experimental conditions can aid in discriminating the oxidation signals corresponding to the interference molecules from the signal specific for THC oxidation.

In some example implementations, absorbent materials may be included that facilitate removal of interference molecules and proteins from the sample before conducting electrochemical measurements. Non-limiting examples of such absorbent materials are magnetic nanoparticles, such as iron oxide nanoparticles (IONPs), which are particles with nanometer dimensions and handled using a magnet.

Referring now to FIG. 1D, a method for eliminating interference molecules in a saliva sample is schematically illustrated. As shown at steps 1 to 5, a saliva sample can be collected in a reservoir with magnetic particles (e.g. magnetic nanoparticles) (step 1, FIG. 1D) capped with a strongly charged polymeric shell.

The functionalization of the surface of the magnetic nanoparticles allows the interaction with polar molecules (e.g. uric acid, bilirubin, glutamate, cortisol, ascorbic acid, and proteins) during a short incubation time, such as, for example, approximately 1-2 minutes, 1-3 minutes, or 1-5 minutes (step 2, FIG. 1D) due to electrostatic forces. Next, the nanoparticles loaded with the interference molecules may be removed with a magnet in seconds (steps 3-4, FIG. 1D). Finally, the THC molecules, which present lower affinity for magnetic nanoparticles surface, are maintained in the “clean” solution (step 4, FIG. 1D) and can be electrochemically detected, according to the example methods disclosed herein (or other rapid THC detection methods), for example, within minutes or even seconds (step 5, FIG. 1D).

### Magnetic-Particle-Based Concentration of Analyte and Electrically Conductive Nanostructure on Electrode

Magnetic nanoparticles are widely used in immunoassays and other biosensing platforms to capture, concentrate, and separate analyte from a matrix for further detection. However, in stark contrast to this conventional use of magnetic beads, the present inventors realized that an improved electrochemical assay could be achieved with the use of carbon nanomaterial/electrocatalyst nanocomposites and/or graphene sheets that that are functionalized with magnetic particles (e.g. magnetic nanoparticles).

As shown in steps 6 and 7 of FIG. 1D, carbon nanomaterial/electrocatalyst nanocomposites and/or graphene nanosheets functionalized with magnetic particles may be initially contacted with the sample in suspension instead of being adherend to the electrode, thereby facilitating the interaction of the carbon nanomaterial/electrocatalyst nanocomposites and/or graphene sheets with THC molecules in solution (optionally in a “washed” solution that is substantially free from interferents, as per steps 1-5 of FIG. 1D). The carbon nanomaterial/electrocatalyst nanocomposites and/or graphene sheets having THC molecules adhered thereto may subsequently be concentrated on the surface of the sensor electrode (e.g. working electrode) by using a magnet, as shown in steps 8-9 of FIG. 1D. Subsequently, with both the carbon nanomaterial/electrocatalyst nanocomposites and/or graphene sheets and the THC analyte in close proximity to the electrode, enhanced electrochemical detection of the THC analyte may be performed according to the example embodiments described above, as shown at step 10 in FIG. 1D.

### Electrochemical Detection of Phenolic Analytes using Electrodes Modified with Carbon Nanomaterial and Electrocatalyst Nanocomposites and/or Graphene Nanosheets

In some example implementations, electrodes modified with carbon nanomaterial and electrocatalyst nanocomposites and/or graphene nanosheets may be employed in assays involving electrical detection, such as, but not limited to, electrochemical assays. In such cases, the carbon nanomaterial and electrocatalyst nanocomposites and/or graphene nanosheets may be deposited onto a working (or sensing) electrode to form a modified electrode. Non-limiting examples of suitable electrical detection assay modalities include electrochemical detection modalities including voltametric sensors, potentiometric sensors, amperometric sensors, and other examples include field-effect-transistor-based sensors, chemiresistive sensors and conductometric sensors.

As demonstrated in the examples below, the present inventors have found that a systematic enhancement in the limit of detection of THC in saliva has been achieved via the use of working electrodes modified with carbon nanomaterial and electrocatalyst nanocomposites and/or graphene nanosheets.

In various example electrochemical detection embodiments, an analyte-specific current peak, uniquely associated with the electrochemical oxidation of a phenolic analyte (such as, but not limited to, THC), is detected. For example, as described above, the THC electrochemical peak that appears during the electrochemistry-based scanning process is to the oxidation of the phenolic group of the THC parent molecule. The intensity of this peak is directly proportional to the concentration of the phenolic analyte, which is recorded by the electrochemical technique.

The limit of detection and dynamic range of a THC electrochemical assay performed using carbon nanomaterial and electrocatalyst nanocomposites and/or graphene nanosheets may be improved or optimized via the control and tuning of one or more parameters of the assay. It will be understood that the specific parameters that yield an optimized assay will be dependent on the specific material system that is employed. For example, different optimal assay read times, pH, deposition methodology, electrochemical parameters, buffer(s), preincubation time, or other assay parameters may exist for different types and/or concentrations of the carbon nanomaterial or the electrocatalyst employed to form a carbon nanomaterial/electrocatalyst nanocomposite.

It will be understood that the present example assays may be implemented to detect analytes in a wide range of sample types. The sample can be a biological sample which can be, without limitation, an ex vivo bodily fluid that can be a non-invasively obtained fluid (saliva, sputum, urine, tears, etc.) or invasively obtained (blood, plasma, cerebral spinal fluid, etc.). In an embodiment, the bodily fluid is an oral fluid. The oral fluid can include saliva, sputum, or a combination thereof. The sample can be used with the sensor described herein without being processed (e.g., an unprocessed sample). In some embodiments, the bodily fluid sample can first be processed before being used with the sensor described herein.

In some example implementations, electrochemical sensors and associated electrochemical detection methods are employed for the rapid detection of THC in saliva. A quantity of saliva suspected of containing THC, such as 0 ng/mL to 1000 ng/mL is contacted with a working electrode modified with carbon nanomaterial and electrocatalyst nanocomposites and/or graphene nanosheets and a voltametric method, such as cyclic voltammetry, is employed to detect an assay signal (e.g. an electrochemical current peak) associated with the electro-oxidation of THC. As noted above, the electrode that is modified by the presence of the carbon nanomaterial/electrocatalyst nanocomposite and/or graphene nanosheets may be a screen-printed electrode (e.g. a carbon screen-printed electrode). In some example implementations, the sample is contacted or mixed with a buffer prior to or upon contact with the working electrode. Non-limiting examples of suitable buffers include a phosphate buffer, borate buffer, and carbonate buffer. Examples of suitable quantities of buffer include, but are not limited to, 10 μL to 50 μL. The buffer may be employed to maintain a selected pH during the assay. Non-limiting examples of a suitable pH include 5 to 10. Examples of suitable potentials for the electrochemical detection of a current perk associated with the electrochemical oxidation of THC include 0.35 V to 0.5 V.

In some example implementations, an electrode modified with a carbon nanomaterial/electrocatalyst nanocomposite and/or graphene nanosheets is electrically-pretreated, after contact with the sample, by applying a pre-conditioning potential for a pre-conditioning time, prior to performing voltametric detection (e.g. detecting a current at a prescribed voltage or detecting a current peak while sweeping an applied voltage). FIG. 2B illustrates the incorporation of a pre-treatment step in the operation of an example electrochemical THC detection device that employs a working electrode modified with a carbon nanomaterial/electrocatalyst nanocomposite and/or graphene nanosheets.

Non-limiting examples of suitable pre-conditioning potentials and pre-conditioning times include 0.1 mV to 0.5 mV at 30 seconds to 120 seconds. This pre-treatment step may assist in the preconcentration of THC molecules on the nanostructures and facilitate electro-oxidation of hydroxyl groups attached to C-1 of THC chemical structure (as shown in FIG. 1A). Moreover, in the case of an electrode modified with graphene nanosheets, the graphene nanosheets may act as a synergistic transducer for the electrochemical signal intensification and aid in the better adsorption of THC on their lattice with simple π-π and electrostatic interactions.

The present inventors have found that when employing a working electrode modified with carbon nanomaterial and electrocatalyst nanocomposites and/or graphene nanosheets for the electrochemical detection of THC, a very rapid assay readout time is feasible while achieving sensitive detection of THC. In some example implementations, the assay signal may be read within 5 minutes, 3 minutes, 2 minutes, or less than or equal to 1 minute of incubation time. In some example implementations, these readout times may be achieved while obtaining a limit of detection for THC in the range of 5 ng/mL to 1000 ng/mL.

As described below, the rapid assay readout time, low limit-of-detection, and long-term stability of the electrodes modified according to the example embodiments described above can be employed to provide a rapid, sensitive, and portable THC testing device that is suitable for roadside testing. Moreover, devices employing electrodes modified with carbon nanomaterial and electrocatalyst nanocomposites and/or graphene nanosheets have been demonstrated to show long-term stability and reproducible electrochemical peaks.

In some example implementations, the multiplexed sensor configurations may be provided that facilitate multiparametric detection and/or detection of two or more different analytes. For example, an electrochemical sensor can be fabricated that can employ a single modified electrode to detect multiple analytes having different peak potentials, or, for example, including two separate modified working electrodes, each being configured for sensing of a different analyte (e.g. THC and ethanol).

As demonstrated below, an electrochemical THC assay may be implemented using electrodes modified with carbon nanomaterial and electrocatalyst nanocomposites and/or graphene nanosheets without substantial interference with from other analytes or interferents, such as methanol, ethanol, or potassium, sodium, nitrogen, magnesium, calcium ions, due to specific electro-oxidation of active THC molecule, achieved by applying a oxidation potential of THC, optionally as well as by utilizing the specific enzymes in the nanocomposite.

A portable reader may be employed to perform rapid electrochemical assays with electrodes modified with carbon nanomaterial and electrocatalyst nanocomposites and/or graphene nanosheets, with a current range in microampere range, using the potential of potentiostat under 100 mV, and with operation under room temperature conditions.

### Devices and Systems for Electrochemical Detection of Phenols

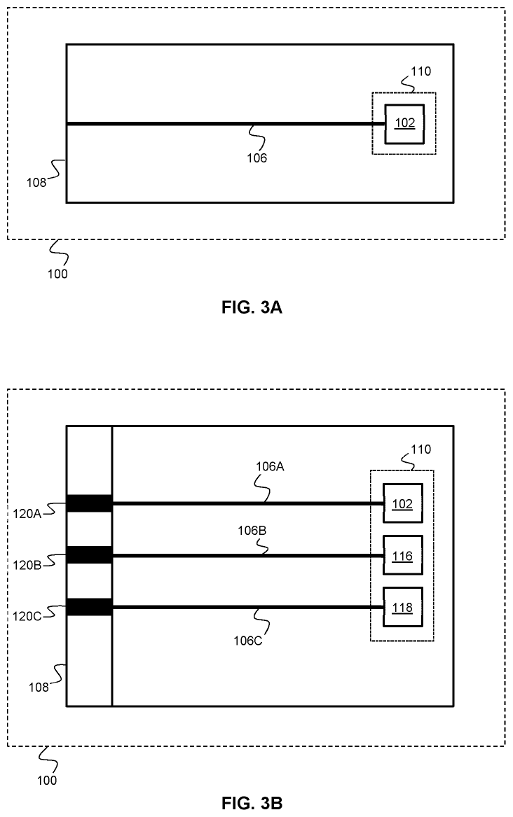

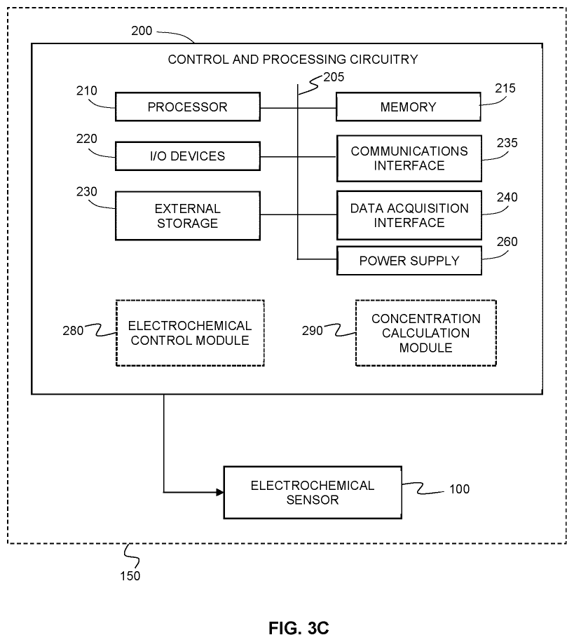

Referring now to FIG. 3C, a system for performing electrochemical detection with a sensor having a working electrode modified with a carbon nanomaterial/electrocatalyst nanocomposite and/or graphene nanosheets is schematically illustrated. The example system includes an electrochemical sensor 100, which may include a modified working electrode, a reference electrode, and a counter electrode.

The electrochemical sensor 100 is operatively coupled to control and processing circuity 200. As shown in the example embodiment illustrated in FIG. 3C, the control and processing circuitry 200 may include a processor 210, a memory 215, a system bus 205, one or more input/output devices 220, and a plurality of optional additional devices such as communications interface 235, external storage 230, data acquisition interface 240 and a power supply 160. The example methods described above can be implemented via processor 210 and/or memory 215. As shown in FIG. 3C, executable instructions represented as electrochemical control module 280 and concentration calculation module 290 are processed by control and processing circuitry 200 to execute instructions for performing one or more of the methods described in the present disclosure, or variations thereof. Such executable instructions may be stored, for example, in the memory 215 and/or other internal storage.

The methods described herein can be partially implemented via hardware logic in processor 210 and partially using the instructions stored in memory 215. Some embodiments may be implemented using processor 210 without additional instructions stored in memory 215. Some embodiments are implemented using the instructions stored in memory 215 for execution by one or more microprocessors. Thus, the disclosure is not limited to a specific configuration of hardware and/or software.

It is to be understood that the example system shown in the figure is not intended to be limited to the components that may be employed in a given implementation. For example, the system may include one or more additional processors. Furthermore, one or more components of control and processing circuitry 200 may be provided as an external component that is interfaced to a processing device. Furthermore, although the bus 205 is depicted as a single connection between all of the components, it will be appreciated that the bus 205 may represent one or more circuits, devices or communication channels which link two or more of the components. For example, the bus 205 may include a motherboard. The control and processing circuitry 200 may include many more or less components than those shown. In some example implementations, some aspects of the example methods described herein, such as the processing of the measured signals to calculate one or more blood pressure measures, may be performed via one or more additional computing devices or systems, such as a mobile computing device connected via a local wireless network (such as Wi-Fi or Bluetooth), and/or a remote server connected over a wide area network.

In some example implementations, the electrochemical sensor 100 is provided on a disposable cartridge that can be removably engaged with the control and processing system 200 for performing electrochemical detection. The control and processing circuity may be housed in a portable device.

The electrochemical sensor 100 may be provided according to a wide variety of formats, including, but not limited to, the example open format shown in FIGS. 3A and 3B, a microfluidic device configuration (optionally including one or more valves that are controllable by the control and processing circuitry 200 when the microfluidic device is engaged with the control and processing circuity), a lateral flow configuration, and a paper-based detection system. In some example implementations, the electrochemical sensor may be reusable component that is integrated with the control and processing circuitry, as schematically shown by 150. The control and processing circuitry may reside, at least in part, on a mobile computing device, such as a mobile phone, that is interfaceable with a reader that is configured to receive an electrically actuate and read an electrochemical assay cartridge.

Some aspects of the present disclosure can be embodied, at least in part, in software, which, when executed on a computing system, transforms an otherwise generic computing system into a specialty-purpose computing system that is capable of performing the methods disclosed herein, or variations thereof. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine-readable media, such as discrete hardware components as large-scale integrated circuits (LSI's), application-specific integrated circuits (ASIC's), or firmware such as electrically erasable programmable read-only memory (EEPROM's) and field-programmable gate arrays (FPGAs).

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

Although many example embodiments of the present disclosure have been described with reference to the enhanced electro-oxidation of THC, it will be understood that a wide variety of phenolic analytes may be detected in the alternative. Non-limiting examples of phenolic analytes include, without limitation, a cannabinoid, an opiate, a neurotransmitter, a hormone, or a derivative thereof. The cannabinoid can be, for example, L,9-tetrahydrocannabinol (THC), 11-hydroxy-A9-tetrahydrocannabinol (11-hydroxy-THC), delta-8-tetrahydrocannabinol (08-THC), 11-nor-9-carboxy-tetrahydrocannabinol (11-nor-9-carboxy-THC), cannabidiol (CBD), cannabinol (CBN), and glucuronic acid conjugated COOH-THC (gluc-COOH-THC), tetrahydrocannabinolic acid (THCA) or metabolites thereof. The opiate can be, for example, morphine as well as metabolites thereof. The neurotransmitter can be, for example, dopamine, serotonin, or metabolites thereof. The hormone can be, without limitation, a steroid hormone such as, for example, estradiol, 7amethylestradiol, or metabolites thereof.

The following examples are presented to enable those skilled in the art to understand and to practice embodiments of the present disclosure. They should not be considered as a limitation on the scope of the disclosure, but merely as being illustrative and representative thereof.

### EXAMPLES

### Example 1: CNT-Electrocatalyst Nanocomposites for Electrochemical Detection of THC

### Materials

Carbon nanotubes (CNTs) were procured from Plasmachem, Germany. The Ferrocene Carboxylic acid was purchased from Santacruz, Canada. Buffer solutions (such as phosphate buffer saline (PBS), borate buffer), Δ9-THC, potassium ferricyanide, dimethylformamide is procured from Sigma-Aldrich from Ontario, Canada. Ultrapure water (18.2 MΩ, Deionized water, DI) is used throughout the experiments. The modified screen-printed electrodes (Fe@CNT/SPE) were used as transducers during the electrochemical measures. In this study, a screen-printed electrodes (SPE) model TE100 purchased from Zensor R&D Co from Taiwan was used. This model presents a carbon working electrode (4 mm diameter), carbon auxiliary, and Ag/AgCl dot as a reference electrode. The Handheld Potentiostat model PalmSens4 was procured from the Palmsens company USA.

### Preparation of CNT-Electrocatalyst Nanocomposites

A highly conductive electroactive composite (Fe@CNT) was prepared by mixing the multiwalled carbon nanotubes (MWCNTs) with ferrocene carboxylic acid molecules and dispersing the mixture of dimethylformamide and DI water (90% DMF:10% DI water) solvents using the ultrasonic bath for 15-30 minutes at 60-80° C. The MWCNTs concentration was kept at 1 mg mL⁻¹, and Ferrocene concentration was maintained at 1.8 mg mL⁻¹ prior to centrifugation. Finally, the Fe@CNT dispersion was centrifuged at 6,000 rpm for 20 minutes. The supernatant was discarded and pellet was resuspended with 1 mg/mL concentration in Solvent (90% DMF:10% DI water) for further electrochemical assay studies and electrode characterization.

### Modification of Electrodes with Nanocomposite

A volume of 5 to 10 μL of the resulting composite (with a concentration of approximately 1 mg/ml) was spread over the working area of SPEs using micropipettes. The resulting modified SPEs were annealed from 120 to 200° C. for 1 to 3 hours inside a hot air oven. All the modified electrodes (mSPE) were thoroughly rinsed with DI water for further experiments. Cyclic voltammetry was used to analyze the property of the modified electrodes.

The nanocomposite was electrodeposited over the surface of SPE by electrochemical technique. Subsequently, ten cycles of cyclic voltammetry (CV) were applied in the −1 to +1 V potential range at a scan rate of 50 mV S⁻¹ for the electrodeposition, and a stability curve was obtained by the potentiostat. Cyclic voltammetry was also employed to analyze the electrochemical properties of the modified electrodes to verify the enhancement in the conduction behaviour of the modified electrodes. The schematic representation of the composite deposited on Fe@CNT/SPE and the electrocatalytic effect of ferricyanide is represented in FIG. 1B.

### Incubation and Electrochemical Surface Pre-treatment Approach for the Enhancement of Sensitivity

It was found that more viscous saliva samples took longer to wet the carbon nanostructures immobilized overlayer on the SPE thoroughly, and an incubation time of 60 seconds was required to ensure complete wetting of the overlayer. This incubation step assured that adequate time was provided for the THC molecules to diffuse towards the modified working SPE. Accordingly, 75 μL of THC sample was pipetted onto the working area of carbon nanostructures embedded SPEs and further incubation was allowed for approximately 60 seconds. This natural mode of incubation provides for better interaction between the THC molecules present in the saliva samples and nanostructures immobilized on the surface of working electrodes.

After incubation, a pretreatment electrochemical step was employed using applying a +0.05V potential for 30 seconds before applying the potential oxidation scan of SWV measurements. This pretreatment step enabled Δ9-THC molecules have increased oxidation potential due to proximity to the working electrode, resulting in further (e.g. complete) oxidization.

As described above with reference to FIG. 1B, the Δ9-THC molecules first oxidize to their initial deprotonated intermediary stage and subsequently form a second intermediate, i.e., phenoxide anion, upon applying oxidation potential. Furthermore, the complete electrooxidation generates phenoxy radicals, which interact with another Δ9-THC molecule to initiate the oxidation of another phenolic structure present in the sample, enhancing the sensitivity of the electrochemical based Δ9-THC assay. This pretreatment step of SPE assists in the preconcentration of Δ9-THC molecules on the nanostructures and causes the electrooxidation of hydroxyl groups attached to C-1 of Δ9-THC chemical structure.

### Label-free Electrochemical Quantification of Cannabis Analyte Δ9-THC

The developed nanocomposite modified electroactive interface of the SPE (Fe-CNT/SPE) was used to detect and quantify varying Δ9-THC concentrations using electrochemical techniques such as square wave voltammetry (SWV) and chronoamperometry.

The 50 μL of standard Δ9-THC solutions in an optimized buffer PBS at pH 7.4 and spiked artificial saliva solutions containing known as well as unknown concentrations of Δ9-THC were subjected to the working electrode area of the sensor electrode.

The samples were incubated for an optimized one minute at room temperature to ensure the proper fast binding with nanostructures and in close proximity with the working electrode. The immobilized nanocomposite on the working area of SPEs allows to amplification of electrochemical signals, enhances surface loading, and minimizes the background noise signals. This facilitates a high signal-to-baseline ratio, aiding in reliable, reproducible, and fast Δ9-THC assay results within 1 minute.

The performance of the modified sensor surfaces was monitored according to the variation in the current response of the prepared electrodes for the quantification of analyte Δ9-THC over a broad range concentration (0 ng/mL to 10,000 ng/mL in optimized buffer and pH). A blank buffer was used as a control baseline current measurement. Further, the spiked sample in simulated saliva with the unknown concentrations of Δ9-THC was tested with Fe-CNT/SPE to evaluate the functional execution of the electrochemical sensor and then calculated the average current response to determine the Δ9-THC sample concentration.

### Cross-reactivity, Validation, and Shelf-life Studies

The Fe@CNT/SPE specificity was evaluated by examining their response against non-specific analyses such as a lactic acid solution, urea, ethanol, and D (+) Glucose. The SWV response to the sensor surface was recorded by inserting and incubating non-specific analytes (potential interferents).

The reproducibility and sensor to sensor variation were also evaluated by repeating Δ9-THC detection experiments, measuring Δ9-THC response five times with the different electrodes toward a fixed analyte concentration (100 ng/mL Δ9-THC).

Additionally, the stability of the Fe@CNT/SPE sensors was also assessed over prolonged storage conditions. Several modified electrodes were kept in refrigeration (4° C.) for this comparison and shelf life study. The electrodes were tested for their sensing response against 100 ng/mL Δ9-THC concentration during regular intervals.

### Characterization of the Nanocomposite Modified SPE

The synthesized ferrocene-CNT nanocomposite has been immobilized to the working surface of SPEs by the dual approach of physisorption and electrodeposition in order to provide a robust surface for the wide range Δ9-THC sensing with high sensitivity.

The nanocomposites are prepared and dispersed by a simple liquid ultrasonic method. Subsequently, the composite characterization is carried out to analyze the morphological and structural properties of the assynthesized CNT-based nanocomposite.

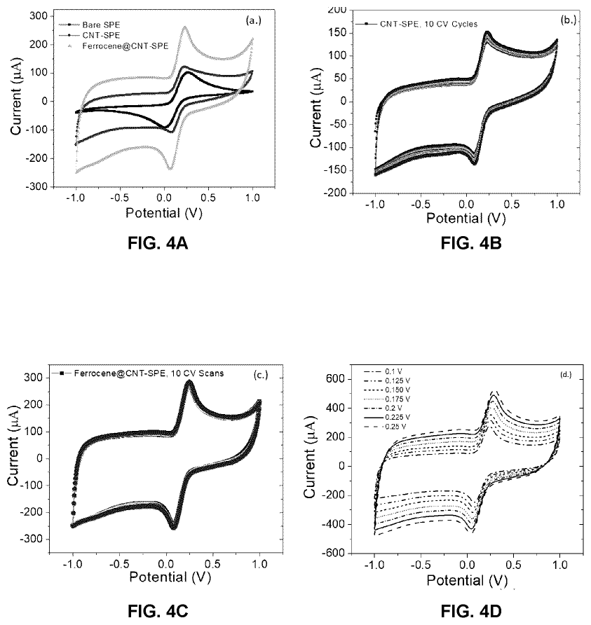

The electrodeposition of nanocomposite over the working electrode of SPE was accomplished using Cyclic Voltammetry (CV) technique. The CV curves of electrodeposited nanocomposite on the working area of the SPE is shown in FIGS. 4A-4D. Furthermore, a highly stable and reproducible CV curve was obtained in the working area of the SPE using 10 repeat cycles, with no difference in redox peaks. CV response was performed in each step of modifying the sensor, as shown in FIGS. 4A-4D. The graphite electrode surface of the SPE interacts robustly with CNT to support the stability of the electrode through π-π bonding, which provides an additional force for the strong interaction between the CNT atomic layer and the carbon-based SPE.

As shown in FIGS. 4A-4D, when compared to pristine SPEs and CNTs, the CV curve of the nanocomposite modified Fe@CNT/SPE is characterized by a larger area of electrical activity, and the peak current shows a significant upward trend. The improvement in the redox reaction of the Fe@CNT/SPE is due to the inherent redox characteristics of the CNT, which is possible because of its high porosity and its specific interface surface as well as due to the electrocatalytically properties of ferrocene.

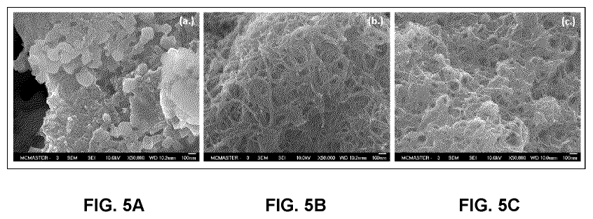

The structure and morphology of modified Fe@CNT SPE electrodes were investigated by the SEM and a SEM micrograph is provided in FIGS. 5A-5C.

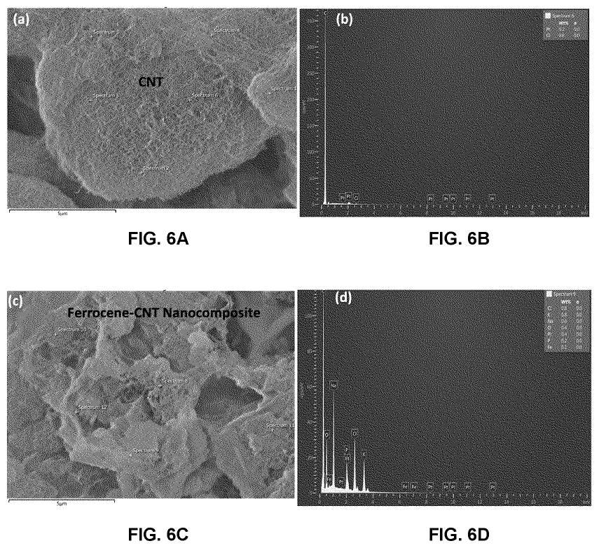

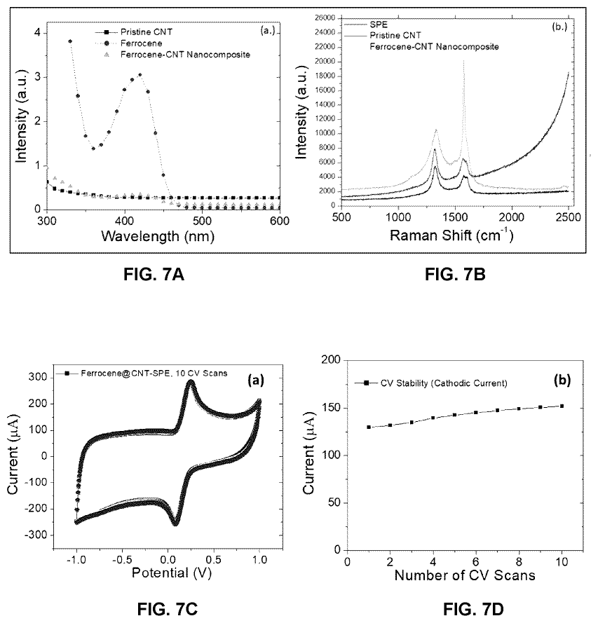

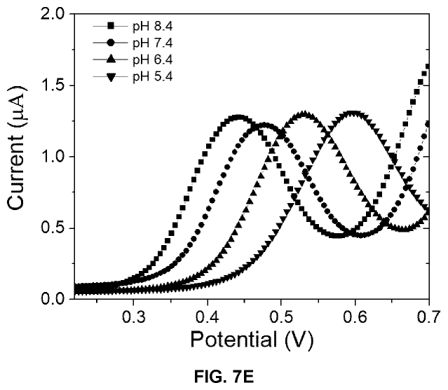

EDX analysis was also performed on the modified electrode surface to demonstrate the purity of the synthesized nanocomposite and that the required surface changes in SPE have been realized. As shown in FIGS. 6A-6D, analysis of the entire area map of the Fe@CNT/SPE shows that the composition of Fe, C's individual element is very evenly distributed on the surface. UV-Vis analysis, as shown in FIG. 7A, demonstrated that the nanocomposite exhibited absorption peaks at ˜330 nm and ˜425 nm, which is attributed to MWCNTs and ferrocene molecules, respectively. Furthermore, as shown in FIG. 7B, Raman spectroscopy was performed to study the intensity, thickness of the structural layer and defects of electrodeposited nanocomposites. The spectra clearly show two characteristic bands of CNT nanostructure, the disordered D band around ˜1330 cm⁻¹ corresponds to the scattering caused by the defects produced in the sp² hybridized two-dimensional hexagonal lattice of carbon structures due to the conjugation of ferrocene molecules, while a crystalline G band around ˜1580 cm⁻¹ is attributed by oscillations of sp² bonding.

FIGS. 7C and 7D show results from repeatability studies of a single modified electrode among multiple CV scans, with FIG. 7C showing results from electrode stability testing of CNT-ferrocene modified screen-printed electrodes using CV, and FIG. 7D plotting the cathodic current over 10 CV cycles to check the stability and irreversibility of nanocomposite modified electrodes.

### Electrochemical Detection of Δ9-THC using a Composite of MWCNT and Ferricyanide in Spiked Buffer Samples

Nanocomposite Fe-CNT modified electrodes were incubated for one minute with varying concentrations of Δ9-THC in standard buffer samples, and subsequently, SWV was applied to quantify the Δ9-THC analyte concentrations.

Optimization studies of buffer, pH, and an ideal incubation time of the Δ9-THC were conducted. FIG. 7E shows results from optimizations studies in which the pH was varied.

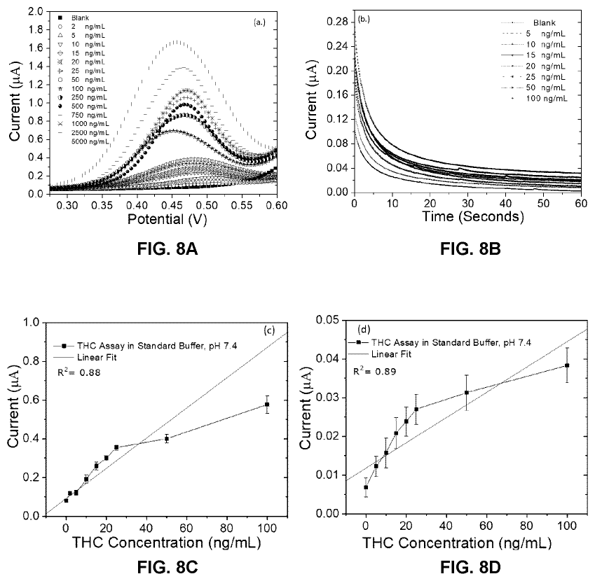

FIGS. 8A-8D present results from studies of the performance of the Fe-CNT-modified-SPE electrochemical sensor using SWV and a peak in the anodic scan was observed at +0.47 V. The results demonstrate a limit of detection of 10 ng/mL and show a linear calibration curve with R2 of ˜0.99 in the range of 0 ng to 25 ng and R² of ˜0.88 in the full broad range 0 ng to 100 ng.

The results also demonstrate that the composite of MWCNTs and ferricyanide increased the oxidation current obtained during the SWV of Δ9-THC. Moreover, the iron (III) ions present in the ferrocyanide acting as electro oxidative catalysts and enhance the oxidation of the Δ9-THC molecule. It is further noted that in implementations in which reference signals are employed that correspond to a PBS buffer, Δ9-THC sample may be detected within a concentration range of from approximately 2 ng/mL to 1000 ng/mL, indicating that a lower detection up to 2 ng/mL.

As demonstrated in these results, under the influence of electrooxidation potential created by the potentiostat, the Δ9-THC molecule gets oxidized very specifically at a particular potential in the range of 0.4V to 0.6V to its oxidized form. This oxidized form of Δ9-THC on the Fe@CNT/SPE electrode provides a unique signal at specific potential created by the potentiostat. The current intensity is then measured by the potentiostat and is correlated to the Δ9-THC concentration present in the sample. The peak arises due to the electrochemical response during the electrooxidation of the phenol functional group in the molecular structure of the Δ9-THC. The intensity of the oxidation current peak is directly proportional to the concentration of Δ9-THC using SWV.

### Electrochemical Detection of Δ9-THC using a Composite of MWCNT and Ferricyanide in Spiked Artificial Saliva Samples

The nanocomposite modified SPE demonstrated excellent redox behavior for the analytical quantification of Δ9-THC in standard buffer solution, representing its applicability for the investigation of other biological fluids. The practicality performance of the Fe@CNT/SPE was further investigated in spiked artificial saliva for the quantification of Δ9-THC in a wide concentration range by employing the SWV. Synthetic Saliva samples were spiked with known Δ9-THC concentrations (significant levels) of Δ9-THC without further dilutions. The Fe@CNT/SPE response was investigated against Δ9-THC spiked artificial saliva samples in the range of 0 to 1000 ng/mL, and the voltammogram was obtained using SWV.

These results highlighted an increase in the current in microampere range with increasing Δ9-THC concentrations attributed to the oxidation of Δ9-THC phenol moieties in the potential range of 0.2 V to 0.5V. Such a broad concentration and linear range highlight this new handheld device's potential applicability for the roadside tests of Δ9-THC.

### Specificity, Reproducibility and Stability Studies

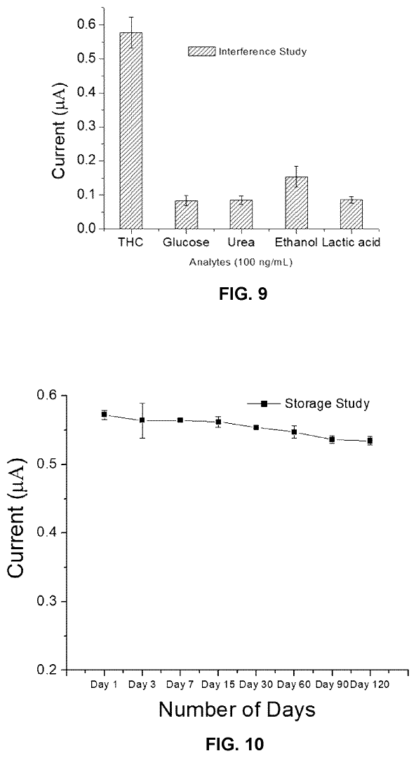

Some structurally related and possible interferents, which commonly found in saliva such as Uric acid, Lactic acid, Glucose, were checked to evaluate the specificity study of Fe@CNT/SPE based electrochemical sensor. The concentration of each analyte kept constant at 100 ng/mL in PBS buffer at pH 7.4. The results, which are shown in FIG. 9, demonstrate that the Fe@CNT/SPE modified electrode is not significantly affected by the presence of these potential interferents. Furthermore, the reproducibility of different Fe@CNT/SPE modified electrodes was tested and all of the modified electrodes yielded a similar sensor response, proving that the proposed Fe@CNT/SPE sensor design has satisfactory reproducibility.

The stability of the Fe@CNT/SPE was also evaluated during 90 days of storage, and results are shown in FIGS. 10A and 10B, a constant sensor response was observed with no significant loss of signal.

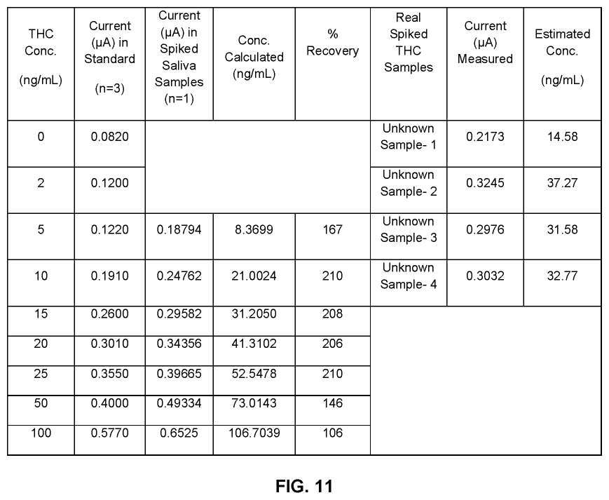

FIG. 11 presents the analysis of standard Buffer, artificial spiked Saliva (containing known THC concentrations) using SWV technique. (Linear Equation y=0.0047x+0.1484 R²=0.8861, calculated from the Calibration plot of THC in Standard PBS Buffer, pH 7.4).

### Example 2: Graphene-Modified Electrodes for Electrochemical Detection of THC

### Materials

Graphene, THC, potassium ferricyanide, dimethylformamide, and buffer such as phosphate buffer saline (PBS), borate buffer are purchased from Sigma-Aldrich from Ontario, Canada. All the chemicals are of analytical grade and used as received without further purification. Ultrapure water (18.2 MΩ, Deionized water, DI) is used throughout the experiments. The modified screen-printed electrodes (eGr/SPE) were used as transducers during the electrochemical measures. In this study, a screen-printed electrodes (SPE) model TE100 purchased from Zensor R&D Co from Taiwan was used. This model presents a carbon working electrode (4 mm diameter), carbon auxiliary, and Ag/AgCl dot as a reference electrode.

### Instruments and Characterization

A scanning electron microscope (Jeol Make) equipped was used for the morphological and structural studies. Measurement of the electrochemical parameters and the subsequent analysis was performed using a Palmsens4 with a 3-electrode connector. Cyclic voltammograms (CVs) were recorded in the −1 to +1V potential range at a scan rate of 50 mV s⁻¹. Square Wave voltammetry (SWV) was conducted from +0.1 V to +1 V at a frequency of 15 Hz, a step potential of 1 mV, and an amplitude of 25 mV. Origin Pro 19 (Origin Lab Corporation, MA, USA) was used for the preparation of graphs. The electrolyte 200 mM PBS (phosphate buffer saline) is used throughout the study for the THC assay development.

### Exfoliation of Graphene Nanostructures

Graphene nanosheets were exfoliated by bath ultrasonication in a 50:50 (DMF:H₂O) dispersion. The concentration of graphene nanosheets was kept at 1 mg mL⁻¹. The dispersion was centrifuged at 6,000 rpm for 30 minutes. The supernatant was used for further studies and characterization. The N, N-Dimethylformamide (DMF)/water in the ratio of 50%:50% (v/v) as an organic mixture was employed to generating a stable graphene dispersion because of its electrochemical steadiness behavior and also compatibility for the THC molecule in case any resides remain after drying.

### Electrodeposition of 2D-Graphene Nanostructures on SPEs (Electrode Preparation)

5 μL of the exfoliated graphene nanosheets suspension (with a concentration of approximately 1 mg/ml) was spread over the working electrode of the screen-printed electrodes (SPEs) and annealed at 120° C. for 1 hour inside a hot air laboratory oven. Subsequently, three reduction electrochemical scans were applied using linear sweep voltammetry (LSV) techniques (at a scan rate of 0.1 V S⁻¹ and step potential of 0.001 V) in the range of 0 to 1.4 V. After the electroreduction step, 10 cycles of the cyclic voltammetry (CV) technique were applied in the −1 to +1 V potential range at a scan rate of 50 mV s⁻¹ for electrodeposition and a stability curve was obtained by the potentiostat.

All the modified electrodes were thoroughly rinsed with DI water for further experiments to remove the unreacted and unbound graphene nanostructures. Cyclic voltammetry technique using the PamSens4 potentiostat was used to analyze the electrochemical features of the modified electrodes to confirm enhancement of the conductive properties of the modified electrodes.

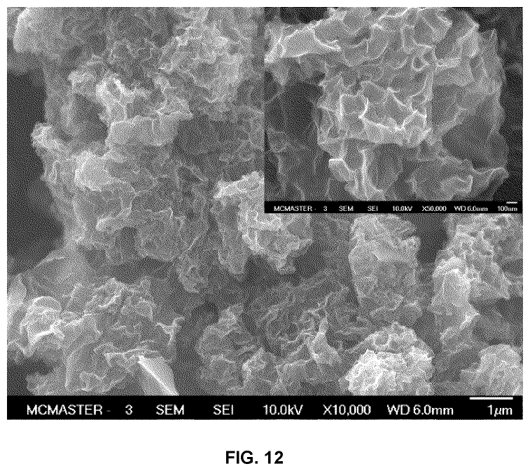

FIG. 12 depicts the SEM image of electrodeposited graphene nanostructures and the formation of the modified eGr/SPE electrode. It is evident from the image that successful electrodeposition of 2D sheet-like nanostructures occurred over the working area of SPE.

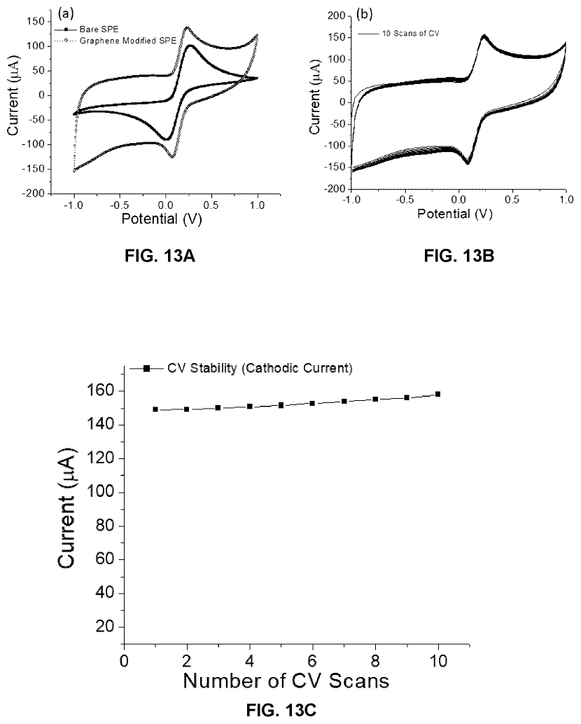

The conducting behavior and electroactive surface area of the bare SPE and eGr/SPE were studied using cyclic voltammetry, and the results are shown in FIG. 13A, with the eGr/SPE modified electrode clearly exhibiting an enhancement in the oxidation and reduction of cathodic-anodic current values due to the high surface area and improved charge transfer. The active surface area of SPE at different stages of fabrication was estimated using the CV equation described by Yola et al. (M. L. Yola, T. Eren and N. Atar, Electrochim. Acta, 2014, 125, 38-47). The surface areas for SPE and eGr/SPE were estimated to be 0.0197 and 0.0355 cm², respectively. The eGr/SPE shown amplification in the electrocatalytic behavior due to high porous 2D structure and increased interfacial surface area.

The current stability and electrocatalytic reproducibility of eGr/SPE were also investigated by performing repetitive redox scans employing the CV technique using a single electrode. Different sets of SPEs were employed to investigate electrode-to-electrode variations. The results, shown in FIG. 13B and FIG. 13C, depict the constant intensity of current without any fluctuations in the peak value due to the strong surface π-π bond between eGr/SPE and bare SPE, which provides a stable and robust electrochemical surface.

### Electrochemical Detection of THC via Graphene-Nanosheet-Modified Electrodes

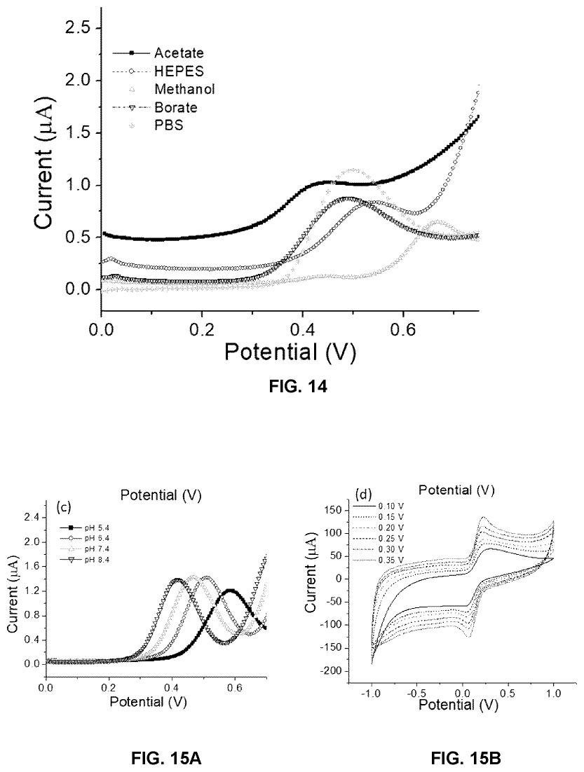

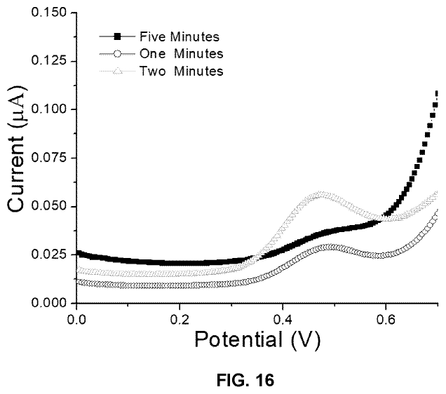

Experiments were performed to demonstrate the quantitation of THC using eGr/SPE modified electrodes. THC solutions were prepared in standard PBS buffer at pH 7.4 and the modified electrodes were tested using square wave voltammetry (SWV), an electrochemical label-free technique. However, prior to such investigations, experiments were performed to achieve optimization of assay parameters such as the buffer composition (FIG. 14), pH (FIG. 15A), Scan rate study (FIG. 15B) and incubation time (FIG. 16) of the THC over the modified surface were conducted.

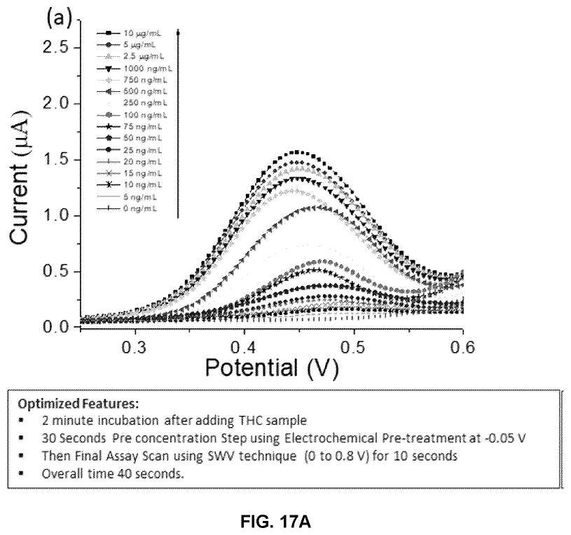

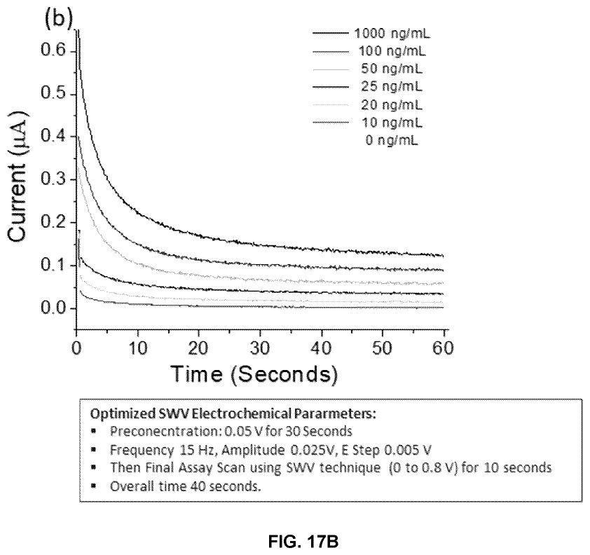

FIGS. 17A and 17B illustrate the performance of an example eGr/SPE-modified electrochemical sensor, using SWV, and a peak in the anodic scan was observed at +0.48 V with a 25 ng/mL limit of detection (LoD=LoB+1.645 (SD low concentration sample)) as well as showing a linear calibration curve with R²=0.99. A specific peak appeared at +0.48 V, and peak height increased with increasing THC concentration.

As can be seen from the figures, these example sensors were able to achieve a nanomolar limit of detection, which is comparable to THC detection thresholds that are currently employed by federal regulations and law enforcement agencies in various parts of the world. The peak obtained using SWV at +0.48 V is attributed to the presence of the phenolic ring in the THC analyte. The control experiment was conducted in the presence of THC, as well as in the absence of THC (using blank PBS buffer solution). The obtained SWV response of the eGr/SPE was recorded as displayed in FIG. 2A.

### Demonstration of Electrochemical THC Detection using Spiked Artificial Saliva

The eGr/SPE modified electrodes demonstrated an excellent redox behavior for the analytical quantification of THC in standard buffer solution, representing its applicability for the investigation of other biological fluids. The practicality performance of the eGr/SPE was further investigated in lab-made spiked artificial saliva for the quantification of THC in a wide concentration range by employing the SWV and chronoamperometric detection. Synthetic Saliva samples were spiked with known THC concentrations (clinically significant levels) of THC without any pretreatment. 50 μL of standard THC solutions in the optimized buffer PBS at pH 7.4 and also spiked artificial saliva solutions containing known as well as unknown concentrations of THC, were exposed to the working electrode area of the modified eGRr/SPE sensor electrode. The sample was left to incubate for an optimized value of approximately 2 minutes at room temperature to ensure the efficient binding with the nanostructure and in close proximity with the modified working electrode.

In some experiments, square Wave voltammetry (SWV) was performed from +0.1 V to +1.00 Vat a step potential of 5 mV, an amplitude of 25 mV, and a frequency of 15 Hz. Furthermore, in some implementations, a surface pretreatment and preconcentration step was performed at 0.05 V for 30 seconds. The preconditioning permits the THC phenolic rings to get placid at the working electrode surface (modified with Graphene) due to electromotive forces. THC phenolic chemical structure in direct contact with the nanostructures, which further get easily oxidized upon applying oxidation potential. Moreover, graphene embedded SPE act as a synergistic transducer for the electrochemical signal intensification and aid in the better adsorption of THC on their lattice with simple π-π and electrostatic interactions.

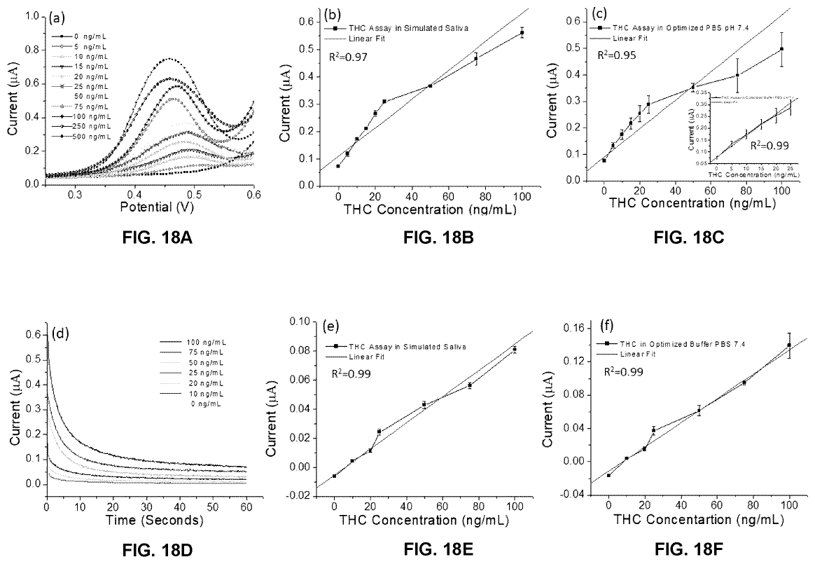

Electrochemical measurements (SWV and chronoamperometric) were recorded to monitor the change in the current response of the Gr/SPE modified electrodes for the quantification of analyte THC over a wide range of analyte concentration (i.e., from 10 ng/mL to 10,00 ng/mL in PBS buffer at the optimized pH). The results of the electrochemical THC sensing assay output based on the SWV technique are shown in FIGS. 18A-18F. Baselines were established by analyzing a blank control sample. To further evaluate the practical performance of the electrochemical sensor, spiked samples with unknown concentrations were tested by the electrochemical sensor, and the average current response was then calculated to determine the THC sample concentration. As a result, the signal-to-noise ratio is high, aiding in reliable, reproducible, and fast THC assay results within 2 minutes.

The recorded current values were correlated with the standard data and equations, as shown in the calibration plots (FIGS. 18A-18F). Based on the obtained results from SWV, the current vs analyte concentration was plotted with 0.99 linear regression fit (R²) value, as shown in FIG. 18B and FIG. 18C. These results highlighted an increase in the current in microampere range with increasing THC concentrations attributed to the oxidation of THC phenol moieties in the potential range of 0.2 V to 0.5V. Such a wide concentration and linear range highlight the potential applicability of this new handheld device for the roadside tests of THC.

The THC concentration in spiked samples was also tested by the chronoamperometric (AD) technique. Based on the voltammetry study, it was observed that an optimal potential +0.48 V (responsible for THC oxidation) is ideal for the chronoamperometric study. The achieved current vs. time as a function of varying THC concentrations are shown in FIG. 18D. The corresponding linear regression fit of current vs. analyte concentration was plotted in FIGS. 18E and 18F. The eGr/SPE surface demonstrated a linear amperometric response with R²˜0.99.

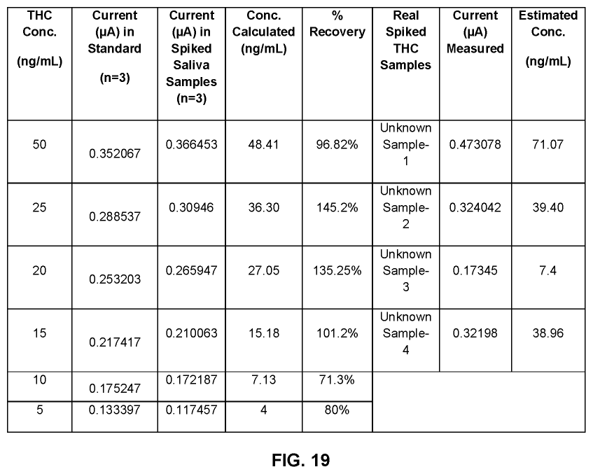

The detailed results of the electrochemical analysis of THC by the SWV technique are given in FIG. 19, which highlights that the electrochemical sensing system proposed in this study was highly useful to reliably analyze the THC concentrations in spiked saliva and standard samples. The obtained results of the SWV and AD technique were cross-validated with the commercial colorimetric based method in both standards, and spiked artificial saliva samples. The present eGr/SPE-based electrochemical detection approach thus appears to be reliable and consistent with the results obtained from existing commercial kits.

### Specificity, Reproducibility and Stability Studies

The specificity of the developed sensor electrodes was evaluated by investigating their response against non-specific analytes, namely ethanol, lactic acid solution, Urea) and glucose (Glu). The SWV response of the sensor surface was recorded after incubating the non-specific analytes. The reproducibility and sensor to sensor variation was also evaluated by repeating the THC detection experiment, measuring THC response five times with the different electrode toward a fixed analyte concentration (100 ng/mL THC in PBS buffer at pH 7.4).

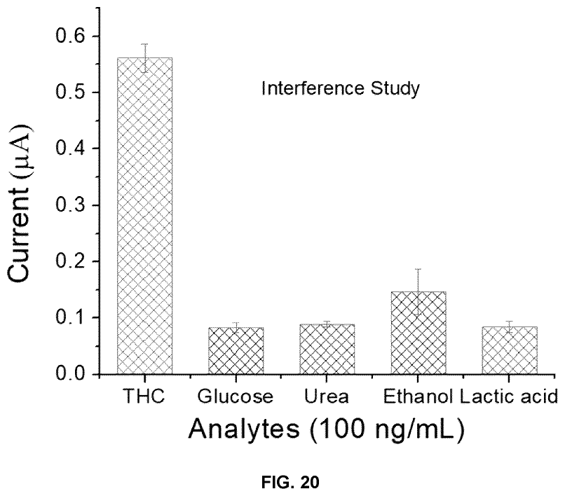

The results of the specificity experiments are presented in FIG. 20, which demonstrates that eGr/SPE is not significantly affected by the presence of these potential interferents. All of the different electrodes yielded a similar sensor response, proving that eGr/SPE electrochemical sensors are can be fabricated with satisfactory reproducibility and performance in the presence of real samples. It was also observed that there was an absence of any additional peaks in the potential range of 0.4 V to 0.5 V. This potential range is only showing a single peak, which is only due to target THC analyte and current intensity changed due to THC, by virtue of the oxidation of phenolic structure in this potential range.

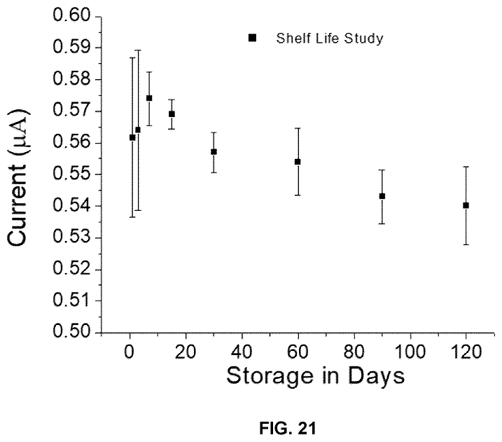

The storage and shelf life of eGr/SPE based electrochemical sensor was also monitored over 120 days. The results, shown in FIG. 21, indicate the capability of the devices to generate THC-responsive electrocurrents for many months.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A method of performing an electrochemical assay to detect an analyte comprising an oxidizable phenolic group, the method comprising:
contacting a sample suspected of containing the analyte with a modified electrode, wherein the modified electrode comprises a nanocomposite, the nanocomposite comprising a carbon nanomaterial and an electrocatalyst;
incubating the sample with the modified electrode;
applying a potential suitable for electrochemically oxidizing the oxidizable phenolic group of the analyte; and
detecting an assay signal associated with electrochemical oxidation of the analyte.

2. The method according to claim 1 wherein the carbon nanomaterial comprises carbon nanotubes.

3. The method according to claim 1 wherein the carbon nanomaterial comprises graphene.

4. The method according to claim 1 wherein the carbon nanomaterial comprises one or more of graphene quantum dots, fullerenes, and carbon nanoribbons.

5. The method according to claim 1 wherein the electrocatalyst comprises ferrocene, ferricyanide, and/or derivatives thereof.

6. The method according to claim 1 wherein the electrocatalyst comprises any one or more of metal oxide frameworks, metal and metal oxide nanoparticles, Prussian Blue nanoparticles, polymer-metal complexes (ruthenium, iron, manganese) nanoparticles, and dendrimers.

7. The method according to claim 1 wherein the assay signal is processed to infer a concentration of the analyte in the sample.

8. The method according to claim 1 wherein the analyte is a cannabinoid or a metabolite thereof.

9. The method according to claim 8 wherein the cannabinoid is delta 9-tetrahydrocannabinol.

10. The method according to claim 1 wherein the analyte is one of an opiate, a neurotransmitter, a hormone, or a metabolite thereof.

11. The method according to claim 1 wherein the sample is saliva.

12. The method according to claim 9 wherein the assay signal is obtained after an incubation delay of less than 3 minutes.

13. The method according to claim 9 wherein the assay signal is obtained after an incubation delay of less than 2 minutes.

14. The method according to claim 9 wherein the assay signal is obtained after an incubation delay of less than or equal to 1 minute.

15. The method according to claim 12 wherein one or more assay parameters of the electrochemical assay are configured such that a limit of detection of the electrochemical assay lies between approximately 2 ng/ml and 10 ng/ml.

16. The method according to claim 1 further comprising applying a pre-conditioning potential to the modified electrode prior to detecting the assay signal.

17. The method according to claim 1 wherein the assay signal is obtained by performing a voltammetric measurement.

18. The method according to claim 1 further comprising, prior to contacting the sample with the modified electrode:
contacting the sample with a suspension comprising capped magnetic particles, the capped magnetic particles comprising a charged polymeric shell, thereby forming a mixture;
incubating the mixture for a time duration sufficient to facilitate adsorption of polar interferents within the sample onto the capped magnetic particles; and
employing a magnetic field to separate the capped magnetic particles from the mixture, thereby reducing a concentration of the polar interferents within the sample.

19. A method of performing an electrochemical assay to detect a cannabinoid analyte, the cannabinoid analyte comprising delta 9-tetrahydrocannabinol or a metabolite thereof, the method comprising:
contacting a saliva sample suspected of containing the cannabinoid analyte with a modified electrode, wherein the modified electrode comprises graphene nanosheets;
incubating the saliva sample with the modified electrode for a time duration of less than 5 minutes;
applying a potential suitable for electrochemically oxidizing the cannabinoid analyte; and
detecting an assay signal associated with electrochemical oxidation of the cannabinoid analyte.

20. The method according to claim 19 further comprising applying a pre-conditioning potential to the modified electrode prior to detecting the assay signal.

21. A method of performing an electrochemical assay to detect an analyte comprising an oxidizable phenolic group, the method comprising:
contacting a sample suspected of containing the analyte with a suspension comprising a magnetic nanocomposite, the magnetic nanocomposite comprising a carbon nanomaterial and magnetic particles, thereby obtaining a mixture;
incubating the mixture;
applying a magnetic field configured to contact the magnetic nanocomposite with a surface of an electrode;
applying a potential to the electrode, the potential being suitable for electrochemically oxidizing the oxidizable phenolic group of the analyte; and
detecting an assay signal associated with electrochemical oxidation of the analyte.

22. The method according to claim 21 wherein the magnetic nanocomposite further comprises an electrocatalyst.

23. The method according to claim 22 wherein the electrocatalyst comprises ferrocene, ferricyanide, and/or derivatives thereof.

24. The method according to claim 21 wherein the carbon nanomaterial comprises carbon nanotubes.

25. The method according to claim 21 wherein the carbon nanomaterial comprises graphene nanosheets.

26. A method of modifying an electrode to incorporate a nanocomposite, the method comprising:
providing suspension comprising the nanocomposite, the nanocomposite comprising nanocomposite comprising a carbon nanomaterial and an electrocatalyst;
drop casting the suspension onto the electrode; and
incorporating the nanocomposite onto the electrode via electrodeposition.

27. An electrochemical sensor for detecting a presence of a cannabinoid analyte in a sample, the cannabinoid analyte comprising delta 9-tetrahydrocannabinol or a metabolite thereof, the electrochemical sensor comprising a working electrode modified with a nanocomposite, said nanocomposite comprising a carbon nanomaterial and an electrocatalyst configured to catalyze electrochemical oxidation of a phenol group of the cannabinoid analyte.

28. The electrochemical sensor according to claim 27 further comprising control and processing circuitry operatively coupled to said working electrode, said control and processing circuitry comprising at least one processor and associated memory, said memory being programmed with instructions executable by said at least one processor for performing operations comprising:
performing a voltametric scan to obtain an assay signal associated with oxidation of a phenolic analyte at said working electrode, the oxidation being catalyzed by said nanocomposite; and
processing the assay signal to infer a concentration of the phenolic analyte in according to calibration data stored in said memory.
